(19) United States
(12) Patent Application Publication
(10) Pub. No.: US 2026/0217661 A1
(43) Pub. Date: Jul. 30, 2026

(54) TREATMENTS FOR PAIN

(71) Applicant: Sevenless Therapeutics Limited, Canterbury Kent (GB)

(72) Inventor: Keith MENEAR, Canterbury Kent (GB)

(21) Appl. No.: 19/573,823

(22) Filed: Mar. 20, 2026

Related U.S. Application Data
(63) Continuation of application No. PCT/GB2024/052457, filed on Sep. 23, 2024.

(30) Foreign Application Priority Data
Sep. 21, 2023 (GB) 2314527.9

(51) Int. Cl. C07D 237/28; A61K 31/502; C07D 403/12; C07D 409/12

(52) U.S. Cl. CPC C07D 237/28; A61K 31/502; C07D 403/12; C07D 409/12

(57) ABSTRACT

The Application describes new SOS1 inhibitors for use in the treatment of pain and cancer.

This application is a bypass continuation of International (PCT) Application No. PCT/GB2024/052457, filed Sep. 23, 2024, which claims the benefit of priority of United Kingdom Application No. 2314527.9, filed on Sep. 21, 2023, the disclosures of each are hereby incorporated by reference as if written herein in their entireties.

The present invention describes compounds that bind to the Son of Sevenless homolog 1 receptor (SOS1) protein thereby inhibiting a cascade pathway, leading to a reduction in pain.

There remains a need for novel treatments for the treatment of pain. Many of the most efficacious and frequently prescribed pain treatments are opioids. These drugs have a high potential for abuse and addiction. However, to date there are no treatments of comparable efficacy to replace them as front-line treatments. To provide a paradigm shift in the treatment of pain requires the identification of new targets and pathways within the body.

This application describes novel SOS1 inhibitors suitable for the treatment of Pain and cancer.

SOS1 inhibitors have recently been identified capable of mediating several conditions:

WO2019/122129 describes benzylamino substituted pyridopyrimidines as SOS1 inhibitors useful in the treatment of cancerous growth in oncology.

WO2018/115380 describes benzylamino substituted quinazolines as SOS1 inhibitors, similarly useful in the treatment of cancerous growth in oncology.

WO2018/172250 describes a genus of 2 methyl quinazolines for use in treating hyper-proliferative diseases.

WO2019/201848 describes a further genus of 2 methyl quinazolines for use in treating hyper-proliferative diseases.

WO2022/207673 teaches the use of SOS1 inhibitors to treat pain

Further SOS1 inhibitors are taught in Proceedings of the National Academy of Sciences of the United States of America (2019), 116(7), 2551-2560.

The present invention provides novel SOS1 inhibitors, novel SOS1 inhibitors for use in the treatment of Pain and novel SOS1 inhibitors for use in the treatment of cancer.

### FIGURES

FIG. 1. NGF signal transduction pathway leading to pain and the clinical drugs that validate the pathway. NGF binds to TrkA and subsequent signal transduction culminates in the nuclear accumulation of diphopshorylated Extracellular signal-regulated kinase (dppERKnuc) in neurons, upregulating pain genes.

FIG. 2. Clinical genetic validation of the target. In NF1, patients have a mutation in the neuronal gap protein (NF1). The mutation causes a loss of function, preventing the normal turnover of GTP on RAS_{GTP} to GDP in turn increasing the concentration of RAS_{GTP} and leading to excess signalling, tumours and pain.

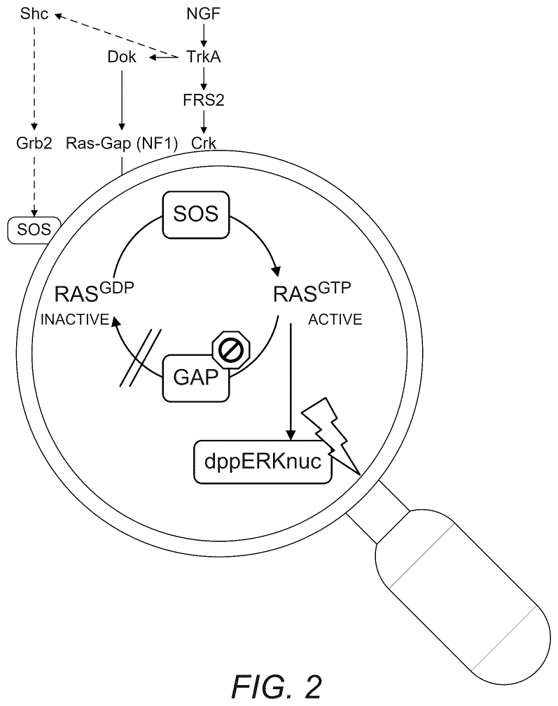

### SUMMARY OF INVENTION

This application describes novel SOS1 inhibitors, their use to treat pain and their use to treat cancer.

The present invention provides compounds of formula (I)

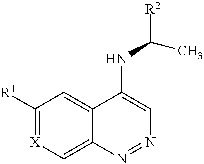

Or a pharmaceutically acceptable salt or solvate thereof, wherein:
X is N or CR⁹
R¹ is selected from O—C₁₋₆ alkyl, O—R¹⁰, O—C₁₋₆ alkyl-R¹⁰ and R¹¹
R² is selected from

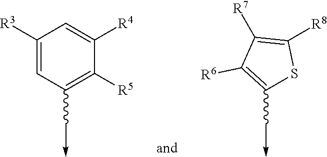

R³ is H or NH₂
R⁴ is CF₃, CF₂CH₂OH, CN
R⁵ is halo
R⁶ is H
R⁷ is H
R⁸ is Ph, ortha substituted by CH₂NHCH₃
R⁹ is H, OCH₃, F and CN
R¹⁰ is 4-6 membered saturated heterocycle containing 1-2 heteroatoms, selected from O, N & S wherein S may also include SO and SO₂ moieties, said heterocycle may also be optionally substituted by C(O)C₁₋₆ alkyl, S(O)₂C₁₋₆ alkyl and C₁₋₆ alkyl.
R¹¹ is 4-6 membered saturated, or partially saturated heterocycle containing 1-2 heteroatoms, selected from O, N & S wherein S may also include SO and SO₂ moieties, said heterocycle may also be optionally substituted by 1-2 groups selected from C(O)C₁₋₆alkyl, N C(O)C₁₋₆ alkyl, OH, an ether linkage to form a bi-cycle and C₁₋₆ alkyl.

The present invention provides SOS1 inhibitors for use in the treatment of Pain.

The present invention provides SOS1 inhibitors for use in the treatment of Cancer.

The present invention provides SOS1 inhibitors for use in the treatment of Dementia.

The present invention provides SOS1 inhibitors for use in the treatment of Parkinsons disease.

The present invention provides novel compounds, uses of the compounds as medicaments, pharmaceutical compositions and pharmaceutical products as set out in the appended claims. Other features of the invention will be apparent from the dependent claims, and the description that follows.

Unless otherwise stated, the following terms used in the specification and claims have the meanings set out below.

The term “comprising” or “comprises” means including the component(s) specified but not to the exclusion of the presence of other components. The term “consisting essentially of” or “consists essentially of” means including the components specified but excluding other components except for materials present as impurities, unavoidable materials present as a result of processes used to provide the components, and components added for a purpose other than achieving the technical effect of the invention. The term “consisting of” or “consists of” means including the components specified but excluding other components.

Whenever appropriate, depending upon the context, the use of the term “comprises” or “comprising” may also be taken to include the meaning “consists essentially of” or “consisting essentially of”, and may also be taken to include the meaning “consists of” or “consisting of”.

The optional features set out herein may be used either individually or in combination with each other where appropriate and particularly in the combinations as set out in the accompanying claims. The optional features for each aspect or exemplary embodiment of the invention, as set out herein are also applicable to all other aspects or exemplary embodiments of the invention, where appropriate. In other words, the skilled person reading this specification should consider the optional features for each aspect or exemplary embodiment of the invention as interchangeable and combinable between different aspects and exemplary embodiments.

References to “treating” or “treatment” include prophylaxis as well as the alleviation of established symptoms of a disease or medical condition. “Treating” or “treatment” of a disease or medical condition therefore includes: (1) preventing or delaying the appearance of clinical symptoms of the disease or medical condition developing in a human that may be afflicted with or predisposed to the disease or medical condition but does not yet experience or display clinical or subclinical symptoms thereof, (2) inhibiting the disease or medical condition, i.e. arresting, reducing or delaying the development of the disease or medical condition, or a relapse thereof (in case of maintenance treatment) or at least one clinical or subclinical symptom thereof, or (3) relieving or attenuating the disease, i.e. causing regression of the disease or medical condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

The term “(C₁₋₆)alkyl” refers to a linear or branched hydrocarbon chain containing 1, 2, 3, 4, 5 or 6 carbon atoms, for example methyl, ethyl, n-propyl, iso-propyl, n-butyl, sec-butyl, tert-butyl and n-pentyl.

The term “halo” refers to one of the halogens, group 17 of the periodic table. In particular, the term refers to fluoro, chloro, bromo and iodo. Suitably, the term refers to fluoro or chloro, most suitably fluoro.

Where a moiety is substituted, it may be substituted at any point on the moiety where chemically possible and consistent with atomic valency requirements. The moiety may be substituted by one or more substituents, e.g. 1, 2, 3 or 4 substituents; optionally there are 1 or 2 substituents on a group. Where there are two or more substituents, the substituents may be the same or different.

Substituents are only present at positions where they are chemically possible, the person skilled in the art being able to decide (either experimentally or theoretically) without undue effort which substitutions are chemically possible.

The phrase “compound of the invention” means those compounds, i.e. of formula (I), that are disclosed herein, both generically and specifically.

### Compounds

The present invention provides a compound of formula (I):

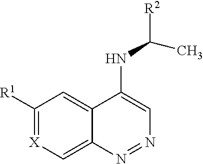

Or a pharmaceutically acceptable salt or solvate thereof, wherein:
X is N or CR⁹
R¹ is selected from O—C₁₋₆ alkyl, O—R¹⁰, O—C₁₋₆ alkyl-R¹⁰ and R¹¹
R² is selected from

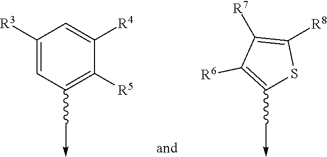

R³ is H or NH₂
R⁴ is CF₃, CF₂CH₂OH, CN
R⁵ is halo
R⁶ is H
R⁷ is H
R⁸ is Ph, ortha substituted by CH₂NHCH₃
R⁹ is H, OCH₃, F and CN
R¹⁰ is 4-6 membered saturated heterocycle containing 1-2 heteroatoms, selected from O, N & S wherein S may also include SO and SO₂ moieties, said heterocycle may also be optionally substituted by C(O)C₁₋₆ alkyl, S(O)₂C₁₋₆ alkyl and C₁₋₆ alkyl.
R¹¹ is 4-6 membered saturated, or partially saturated heterocycle containing 1-2 heteroatoms, selected from O, N & S wherein S may also include SO and SO₂ moieties, said heterocycle may also be optionally substituted by 1-2 groups selected from C(O)C₁₋₆alkyl, N C(O)C₁₋₆ alkyl, OH, an ether linkage to form a bi-cycle and C₁₋₆ alkyl.

In a suitable embodiment, the 4-6 membered saturated heterocycle described in R10 are cyclohexane, 4-hydroxypiperidin-1-yl, pyrrolidine, piperazine, tetrahydrofuran, morpholine, azetidine, tetrahydro-2H-thiopyran 1,1-dioxide, all optionally substituted as described herein.

In a suitable embodiment, the 4-6 membered saturated, or partially saturated heterocycle are described in R11 are cyclohexane, 4-hydroxypiperidin-1-yl, pyrrolidine, dihydropyridine, piperazine, tetrahydrofuran, morpholine, azetidine, tetrahydro-2H-thiopyran 1,1-dioxide all optionally substituted as described herein.

In a suitable embodiment R² is

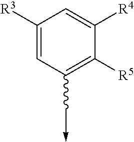

R³ is H or NH₂
Suitably R³ is H
R⁴ is CF₃, CF₂CH₂OH, CN
Suitably R⁴ is CF₃
R⁵ is halo
Suitably R⁵ is F

In another suitable embodiment R² is

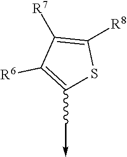

R⁶ is H
R⁷ is H
R⁸ is Ph, ortha substituted by CH₂NHCH₃
Suitably, R¹ is selected from: O—C₁₋₆ alkyl
O—R¹⁰
O—C₁₋₆ alkyl-R¹⁰
R¹¹
Wherein R¹⁰ is

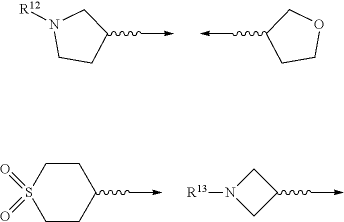

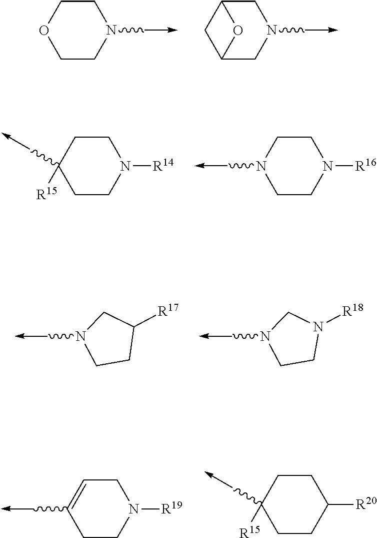

is selected from C(O)C₁₋₆ alkyl, C₁₋₆ alkyl, H
R¹³ is selected from C(O)C₁₋₆ alkyl, C₁₋₆ alkyl, H
R¹⁴ is selected from C(O)C₁₋₆ alkyl, NC(O)C₁₋₆ alkyl, OH and C₁₋₆ alkyl.
R¹⁵ is OH
R¹⁶ is selected from C(O)C₁₋₆ alkyl, NC(O)C₁₋₆ alkyl, OH and C₁₋₆ alkyl.
R¹⁷ is selected from C(O)C₁₋₆ alkyl, NC(O)C₁₋₆ alkyl, OH and C₁₋₆ alkyl.
R¹⁸ is selected from C(O)C₁₋₆ alkyl, NC(O)C₁₋₆ alkyl, OH and C₁₋₆ alkyl.
R¹⁹ is selected from C(O)C₁₋₆ alkyl, NC(O)C₁₋₆ alkyl, OH and C₁₋₆ alkyl.
R²⁰ is selected from C(O)C₁₋₆ alkyl, NC(O)C₁₋₆ alkyl, OH and C₁₋₆ alkyl.

Suitably R¹ is selected from:
O—C₁₋₆ alkyl
O—R¹⁰
O—C₁ alkyl-R¹⁰
R¹¹
Wherein R¹⁰ is

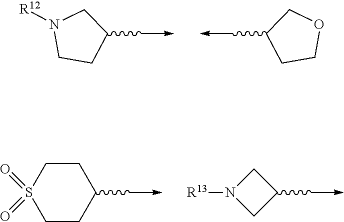

Wherein R₁₁ is

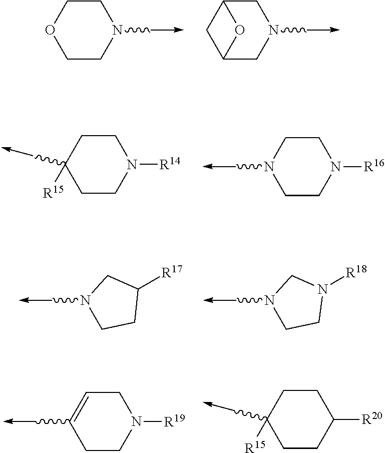

Wherein
R¹² is selected from C(O)CHs, OH₃ and H.
R¹³ is selected from C(O)CHs
R¹⁴ is selected from C(O)C₁₋₆ alkyl.
R¹⁵ is OH
R¹⁶ is selected from C(O)C₁₋₆ alkyl.
R¹⁷ is selected from NC(O)C₁₋₆ alkyl.
R¹⁸ is selected from C(O)C₁₋₆ alkyl.
R¹⁹ is selected from C(O)C₁₋₆ alkyl.
R²⁰ is selected from C(O)C₁₋₆ alkyl, NC(O)C₁₋₆ alkyl, OH and C₁₋₆ alkyl.

In a suitable embodiment X is N or CR⁹, wherein R⁹ is H.

In a suitable embodiment X is CR⁹, wherein R⁹ is H.

Suitably C₁₋₆ alkyl in all embodiments includes methyl, ethyl, propyl, butyl, pentyl and hexyl moieties, straight chain or branched.

In a suitably C₁₋₆ alkyl is methyl, ethyl or propyl, C₁₋₃ alkyl

Suitably C₁₋₆ alkyl is methyl

A suitable pharmaceutically acceptable salt of a compound of formula (I) is for example an acid-addition salt, such as an acid-additional salt with hydrochloric acid, citric acid, tartaric acid and fumaric acid (particularly hydrochloric acid). An acid-addition salt may be obtained, for example, by reaction of a compound of formula (I) with a suitable acid (such as hydrochloric acid, citric acid, tartaric acid and fumaric acid) using a conventional procedure.

A pharmaceutically acceptable salt may alternatively be formed by converting one salt of a compound of the invention to another by reaction with an appropriate acid or base, or by means of a suitable ion exchange column.

The preparation of a pharmaceutically acceptable salt is typically conducted in solution. The resulting salt may precipitate out and be collected by filtration or may be recovered by evaporation of the solvent.

The compounds of formula (I) may form salts in situ under physiological conditions, for example when used as a medicament.

It is to be understood that the compounds of formula (I) may exist in solvated or unsolvated forms, such as for example hydrated forms. The invention encompasses all pharmaceutically acceptable solvated forms.

It is to be understood that, insofar as certain of the compounds of formula (I) defined herein may exist in optically active or racemic forms by virtue of one or more asymmetric carbon atoms, the invention includes within its definition any such optically active or racemic forms.

It is to be understood that the invention relates to compounds of formula (I) that are isotopically-labelled (i.e. radio-labelled). In such compounds, one or more atoms are replaced by an atom having an atomic mass or mass number different from the atomic mass or mass number typically found in nature. Examples of radionucleotides that can be included in the compounds of the invention include ²H (also written as “D” for deuterium), ³H (also written as “T” for tritium), ¹¹C, ¹³C, ¹⁴C, ¹⁵O, ¹⁷O, ¹⁸O, ¹⁸F and the like. The particular radionucleotide used will depend on the specific application of the radio-labelled compound.

Some compounds of the invention may contain one or more chiral centres and may therefore exist as stereoisomers. Stereoisomers may be separated using conventional techniques, such as chromatography or fractional crystallisation. The enantiomers may be isolated by separation of a racemate for example by fractional crystallisation, resolution or HPLC. The diastereoisomers may be isolated by separation by virtue of the different physical properties of the diastereoisomers, for example by fractional crystallisation, HPLC or flash chromatography. Alternatively, particular stereoisomers may be prepared by chiral synthesis from chiral starting materials under conditions that will not cause racemisation or epimerisation, by derivatisation with a chiral reagent or by asymmetric catalytic synthesis. When a specific stereoisomer is isolated, it is suitably isolated substantially free of other stereoisomers, for example containing less than 20%, such as less than 10%, particularly less than 5%, by weight of other stereoisomers.

It is to be understood that the compounds of formula (I) may exhibit polymorphism and the invention encompasses all such forms.

A compound of formula (I), or a pharmaceutically acceptable salt or solvate thereof may be prepared by any process known to be applicable to the preparation of chemically related compounds. Such processes, when used to prepare a compound of formula (I) or a pharmaceutically acceptable salt or solvate thereof are provided as a further feature of the present invention and are illustrated by the following representative process variants. Necessary starting materials are commercially available or may be prepared by standard procedures of organic chemistry within the ordinary skill of an organic chemist, for example as described in conjunction with the following representative processes and within the accompanying examples.

The compounds of the present invention may be prepared using commercially available reagents and intermediates in the synthetic methods and reaction schemes described herein or may be prepared using other reagents and conventional methods well known to those skilled in the art.

For instance, intermediates for preparing compounds of Template I of the present invention may be prepared according to the General Reaction Scheme I.

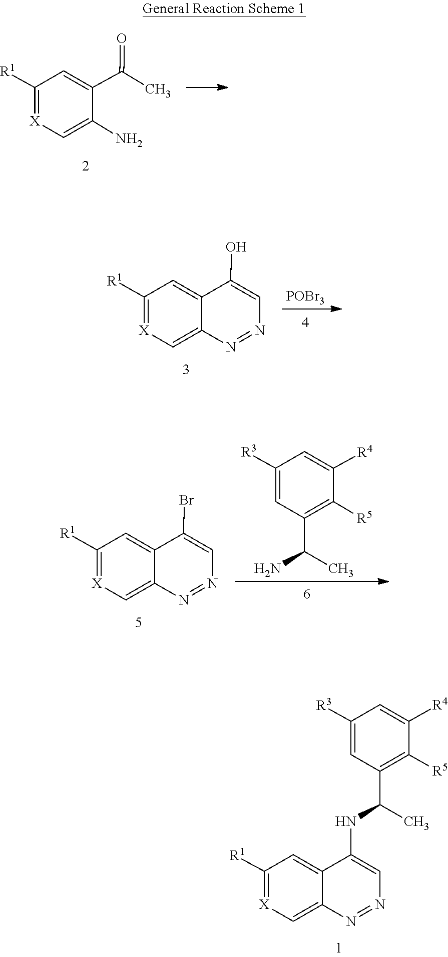

Compound 2 is reacted with sodium nitrite under acidic conditions to give Compound 3 treatment with a bromination reagent such as POBr3 4 then gives Compound 5. Reaction of Compound 5 with a suitable amine such as intermediate 6 by either nucleophilic substitution or metal catalysed reaction provides the title Compound 1.

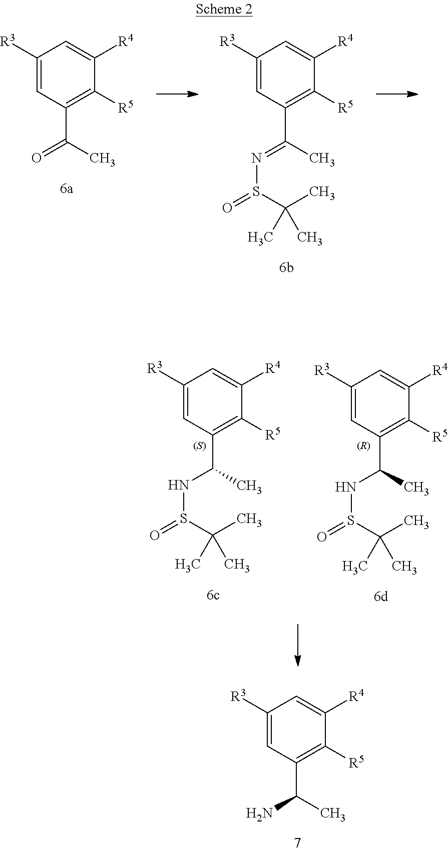

The required benzylic amines represented by Compound 6 can be prepared from aromatic aldehydes such as Compound 6a. There follows formation of a chiral sulfimine 6b followed by reduction to give a diastereomeric mixture of Compounds 6c and 6d that can be separated. The desired diastereoisomer 6d is then taken to the next step where sulphamide cleavage is performed to give the title intermediate 6.

The compounds of the present invention are suitable for use as a medicament.

The present invention also provides for the compounds of the present invention for use in treating pain.

The SOS1 inhibitors have numerous advantages as a pain treatment; they don't have the addiction potential of opiates and they show great efficacy. They also don't appear to have the side effects that make tanezumab and other anti-NGFs almost impossible to use at therapeutically effective doses.

The SOS1 compounds of the present invention also benefit from a high degree of selectivity for SOS1 inhibitors over other targets, boosting efficacy, improving safety and minimising off target effects which can be a source of side effects in patients.

Suitably, the SOS1 inhibitors of the present invention show selectivity of greater than or equal to 100 fold over one or more of the following targets: MEK 1, MEK 2, TrkA kinase, TrkB kinase, TrkC kinase, C-Raf, B-Raf, PI3 kinase, AKT and ERK.

When determining whether a compound of the present invention has a selectivity of more than a 100 fold for SOS1 over another target the following assays and methods may be used:

MEK 1 and 2 can be assayed using MEK assay kit, product code CS0490, Sigma, St Louis, USA.

Trk receptor kinase activity can be assayed as described in Wang et al, Curr Chem Genomics. 2008; 1: 27-33.

B-Raf can be assayed using the B-Raf Kinase Assay Kit, product code 17-359, Sigma, St Louis, USA.

C-Raf can be assayed using the BPS bioscience assay kit catalogue number 79570, San Diego, CA 92121. United States.

PI3 kinase can be assayed via the method described by Fry, Methods Mol Biol, 2009; 462:345-62.

AKT can be assayed using the abcam kit Akt Kinase Activity Assay Kit (ab139436), abcam plc, Cambridge, USA.

ERK can be assayed using the Promega ERK2 kinase kit, catalogue number V1961, Promega corporation, Madison, USA.

The compounds PK makes them particularly suitable for use in the present invention.

The compounds have good bioavailability, permeability, solubility, stability and numerous other qualities associated with a druggable molecule.

Without being bound by theory, its believed that SOS1 inhibitors act to treat pain in the following way:

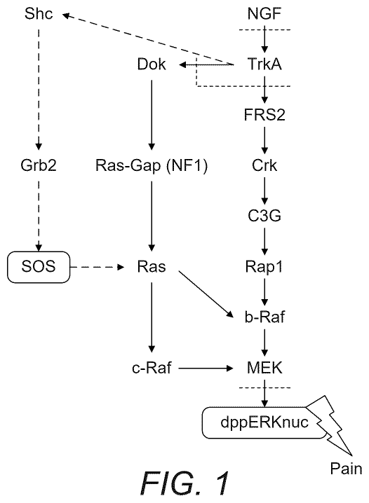

Nerve growth factor (NGF) is a protein that binds to the NGF receptor (TrkA), leading to the upregulation of genes involved in nociception. NGF is known to be an important contributor to the development of chronic pain. The NGF binding to TrkA and subsequent signal transduction culminates in the nuclear accumulation of diphopshorylated Extracellular signal-regulated kinase (dppERKnuc) in neurons, upregulating pain genes, as shown in FIG. 1.

Levels of SOS1 & molecules influenced by it downstream such as Ras feed into this cascade, with greater levels of SOS and RAS leading to higher levels of bRAF and MEK, leading to the accumulation of diphopshorylated Extracellular signal-regulated kinase (dppERKnuc), leading to higher levels of Pain. By inhibiting this pathway it is possible to control the formation of RAS GTP. By lowering levels of RAS GTP, pain is reduced.

The compounds are suitable for use in the treatment of pain.

The compounds provide a method for treating pain.

The term pain includes but is not limited to: acute pain; chronic pain; inflammatory pain; nociceptive pain; neuropathic pain; hyperalgesia; allodynia; central pain; cancer pain; post-operative pain; visceral pain; musculo-skeletal pain; heart or vascular pain; head pain including migraine; orofacial pain, including dental pain; and back pain.

In more detail, suitable pain for treatment includes but is not limited to:
(a) acute pain and/or spontaneous pain,
(b) chronic pain and or on-going pain,
(c) inflammatory pain including any one of arthritic pain, pain resulting from osteoarthritis or rheumatoid arthritis, resulting from inflammatory bowel diseases, psoriasis and eczema
(d) nociceptive pain,
(e) neuropathic pain, including painful diabetic neuropathy or pain associated with post-herpetic neuralgia,
(f) hyperalgesia,
(g) allodynia,
(h) central pain, central post-stroke pain, pain resulting from multiple sclerosis, pain resulting from spinal cord injury, or pain resulting from Parkinson's disease or epilepsy,
(i) cancer pain,
(j) post-operative pain,
(k) visceral pain, including digestive visceral pain and non-digestive visceral pain, pain due to gastrointestinal (GI) disorders, pain resulting from functional bowel disorders (FBD), pain resulting from inflammatory bowel diseases (IBD), pain resulting from dysmenorrhea, pelvic pain, cystitis, interstitial cystitis or pancreatitis,
(l) musculo-skeletal pain, myalgia, fibromyalgia, spondylitis, sero-negative (non-rheumatoid) arthropathies, non-articular rheumatism, dystrophinopathy, Glycogenolysis, polymyositis, pyomyositis,
(m) heart or vascular pain, pain due to angina, myocardical infarction, mitral stenosis, pericarditis, Raynaud's phenomenon, scleredoma, scleredoma or skeletal muscle ischemia,
(n) head pain including migraine, migraine with aura, migraine without aura cluster headache, tension-type headache.
(o) orofacial pain, including dental pain, temporomandibular myofascial pain or tinnitus, or
(p) back pain, bursitis, menstrual pain, migraine, referred pain, trigeminal neuralgia, hypersensitisation, pain resulting from spinal trauma and/or degeneration or stroke.

Treatment of pain includes, but is not limited to, preventing, ameliorating, controlling, reducing incidence of, or delaying the development or progression of pain.

Particularly suitable pain indications include Osteoarthritis and cancer pain.

In another embodiment a suitable indication is osteoarthritis.

According to another aspect of the invention there is provided the compounds of the present invention for separate, sequential or simultaneous use in a combination combined with a second pharmacologically active compound. Suitably the second pharmacologically active compound of the combination may include but is not limited to;
an opioid analgesic, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine or pentazocine;
a nonsteroidal antiinflammatory drug (NSAID), e.g. aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, meloxicam, nabumetone, naproxen, nimesulide, nitroflurbiprofen, olsalazine, oxaprozin, phenylbutazone, piroxicam, sulfasalazine, sulindac, tolmetin or zomepirac;
a barbiturate sedative, e.g. amobarbital, aprobarbital, butabarbital, butabital, mephobarbital, metharbital, methohexital, pentobarbital, phenobartital, secobarbital, talbutal, theamylal or thiopental;
a benzodiazepine having a sedative action, e.g. chlordiazepoxide, clorazepate, diazepam, flurazepam, lorazepam, oxazepam, temazepam or triazolam;
an H₁ antagonist having a sedative action, e.g. diphenhydramine, pyrilamine, promethazine, chlorpheniramine or chlorcyclizine;
a sedative such as glutethimide, meprobamate, methaqualone or dichloralphenazone;
a skeletal muscle relaxant, e.g. baclofen, carisoprodol, chlorzoxazone, cyclobenzaprine, methocarbamol or orphrenadine;
an NMDA receptor antagonist, e.g. dextromethorphan ((+)-3-hydroxy-N-methylmorphinan) or its metabolite dextrorphan ((+)-3-hydroxy-N-methylmorphinan), ketamine, memantine, pyrroloquinoline quinine, cis-4-(phosphonomethyl)-2-piperidinecarboxylic acid, budipine, EN-3231 (MorphiDex®, a combination formulation of morphine and dextromethorphan), topiramate, neramexane or perzinfotel including an NR2B antagonist, e.g. ifenprodil, traxoprodil or (−)-(R)-6-(2-[4-(3-fluorophenyl)-4-hydroxy-1-piperidinyl]-1-hydroxyethyl-3,4-dihydro-2(1H)-quinolinone;
an alpha-adrenergic, e.g. doxazosin, tamsulosin, clonidine, guanfacine, dexmetatomidine, modafinil, or 4-amino-6,7-dimethoxy-2-(5-methane-sulfonamido-1,2,3,4-tetrahydroisoquinol-2-yl)-5-(2-pyridyl) quinazoline;
a tricyclic antidepressant, e.g. desipramine, imipramine, amitriptyline or nortriptyline;
an anticonvulsant, e.g. carbamazepine, lamotrigine, topiratmate or valproate;
a tachykinin (NK) antagonist, particularly an NK-3, NK-2 or NK-1 antagonist, e.g. (αR,9R)-7-[3,5-bis(trifluoromethyl)benzyl]-8,9,10,11-tetrahydro-9-methyl-5-(4-methylphenyl)-7H-[1,4]diazocino[2,1-g][1,7]-naphthyridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]-methyl]-1,2-dihydro-3H-1,2,4-triazol-3-one (MK-869), aprepitant, lanepitant, dapitant or 3-[2-methoxy-5-(trifluoromethoxy)phenyl]-methylamino]-2-phenylpiperidine (2S,3S);
a muscarinic antagonist, e.g oxybutynin, tolterodine, propiverine, tropsium chloride, darifenacin, solifenacin, temiverine and ipratropium;
a COX-2 selective inhibitor, e.g. celecoxib, rofecoxib, parecoxib, valdecoxib, deracoxib, etoricoxib, or lumiracoxib;
a coal-tar analgesic, in particular paracetamol;
a neuroleptic such as droperidol, chlorpromazine, haloperidol, perphenazine, thioridazine, mesoridazine, trifluoperazine, fluphenazine, clozapine, olanzapine, risperidone, ziprasidone, quetiapine, sertindole, aripiprazole, sonepiprazole, blonanserin, iloperidone, perospirone, raclopride, zotepine, bifeprunox, asenapine, lurasidone, amisulpride, balaperidone, palindore, eplivanserin, osanetant, rimonabant, meclinertant, Miraxion® or sarizotan;
a vanilloid receptor agonist (e.g. resinferatoxin) or antagonist (e.g. capsazepine);
a beta-adrenergic such as propranolol;
a local anaesthetic such as mexiletine;
a corticosteroid such as dexamethasone;
a 5-HT receptor agonist or antagonist, particularly a 5-HT_{1B/1D} agonist such as eletriptan, sumatriptan, naratriptan, zolmitriptan or rizatriptan;
a 5-HT_{2A} receptor antagonist such as R(+)-alpha-(2,3-dimethoxy-phenyl)-1-[2-(4-fluorophenylethyl)]-4-piperidinemethanol (MDL-100907);
a cholinergic (nicotinic) analgesic, such as ispronicline (TC-1734), (E)-N-methyl-4-(3-pyridinyl)-3-buten-1-amine (RJR-2403), (R)-5-(2-azetidinylmethoxy)-2-chloropyridine (ABT-594) or nicotine;
Tramadol®;
a PDEV inhibitor, such as 5-[2-ethoxy-5-(4-methyl-1-piperazinyl-sulphonyl)phenyl]-1-methyl-3-n-propyl-1,6-dihydro-7H-pyrazolo[4,3-d]pyrimidin-7-one (sildenafil), (6R,12aR)-2,3,6,7,12,12a-hexahydro-2-methyl-6-(3,4-methylenedioxyphenyl)-pyrazino[2′,1′:6,1]-pyrido[3,4-b]indole-1,4-dione (IC-351 ortadalafil), 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil), 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one, 5-(5-acetyl-2-propoxy-3-pyridinyl)-3-ethyl-2-(1-isopropyl-3-azetidinyl)-2,6-dihydro-7H-pyrazolo[4,3-d]pyrimidin-7-one, 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, 4-[(3-chloro-4-methoxybenzyl)amino]-2-[(2S)-2-(hydroxymethyl)pyrrolidin-1-yl]-N-(pyrimidin-2-ylmethyl)pyrimidine-5-carboxamide, 3-(1-methyl-7-oxo-3-propyl-6,7-dihydro-1H-pyrazolo[4,3-d]pyrimidin-5-yl)-N-[2-(1-methylpyrrolidin-2-yl)ethyl]-4-propoxybenzenesulfonamide;
a cannabinoid;
metabotropic glutamate subtype 1 receptor (mGluR1) antagonist;
a serotonin reuptake inhibitor such as sertraline, sertraline metabolite demethylsertraline, fluoxetine, norfluoxetine (fluoxetine desmethyl metabolite), fluvoxamine, paroxetine, citalopram, citalopram metabolite desmethylcitalopram, escitalopram, d,l-fenfluramine, femoxetine, ifoxetine, cyanodothiepin, litoxetine, dapoxetine, nefazodone, cericlamine and trazodone;
a noradrenaline (norepinephrine) reuptake inhibitor, such as maprotiline, lofepramine, mirtazepine, oxaprotiline, fezolamine, tomoxetine, mianserin, buproprion, buproprion metabolite hydroxybuproprion, nomifensine and viloxazine (Vivalan®), especially a selective noradrenaline reuptake inhibitor such as reboxetine, in particular (S,S)-reboxetine;
a dual serotonin-noradrenaline reuptake inhibitor, such as venlafaxine, venlafaxine metabolite O-desmethylvenlafaxine, clomipramine, clomipramine metabolite desmethylclomipramine, duloxetine, milnacipran and imipramine;
an inducible nitric oxide synthase (iNOS) inhibitor such as S-[2-[(1-iminoethyl)amino]ethyl]-L-homocysteine, S-[2-[(1-iminoethyl)-amino]ethyl]-4,4-dioxo-L-cysteine, S-[2-[(1-iminoethyl)amino]ethyl]-2-methyl-L-cysteine, (2S,5Z)-2-amino-2-methyl-7-[(1-iminoethyl)amino]-5-heptenoic acid, 2-[[(1R,3S)-3-amino-4-hydroxy-1-(5-thiazolyl)-butyl]thio]-5-chloro-3-pyridinecarbonitrile; 2-[[(1R,3S)-3-amino-4-hydroxy-1-(5-thiazolyl)butyl]thio]-4-chlorobenzonitrile, (2S,4R)-2-amino-4-[[2-chloro-5-(trifluoromethyl)phenyl]thio]-5-thiazolebutanol, 2-[[(1R,3S)-3-amino-4-hydroxy-1-(5-thiazolyl) butyl]thio]-6-(trifluoromethyl)-3 pyridinecarbonitrile, 2-[[(1R,3S)-3-amino-4-hydroxy-1-(5-thiazolyl)butyl]thio]-5-chlorobenzonitrile, N-[4-[2-(3-chlorobenzylamino)ethyl]phenyl]thiophene-2-carboxamidine, or guanidinoethyldisulfide;
an acetylcholinesterase inhibitor such as donepezil;
a prostaglandin E₂ subtype 4 (EP4) antagonist such as N-[((2-[4-(2-ethyl-4,6-dimethyl-1H-imidazo[4,5-c]pyridin-1-yl)phenyl]ethyl)amino)-carbonyl]-4-methylbenzenesulfonamide or 4-[(1S)-1-({[5-chloro-2-(3-fluorophenoxy)pyridin-3-yl]carbonyl}amino)ethyl]benzoic acid;
a leukotriene B4 antagonist; such as 1-(3-biphenyl-4-ylmethyl-4-hydroxy-chroman-7-yl)-cyclopentanecarboxylic acid (CP-105696), 5-[2-(2-Carboxyethyl)-3-[6-(4-methoxyphenyl)-5E-hexenyl]oxyphenoxy]-valeric acid (ONO-4057) or DPC-11870,
a 5-lipoxygenase inhibitor, such as zileuton, 6-[(3-fluoro-5-[4-methoxy-3,4,5,6-tetrahydro-2H-pyran-4-yl])phenoxy-methyl]-1-methyl-2-quinolone (ZD-2138), or 2,3,5-trimethyl-6-(3-pyridylmethyl),1,4-benzoquinone (CV-6504);
a sodium channel blocker, such as lidocaine; or
a 5-HT3 antagonist, such as ondansetron;
   and the pharmaceutically acceptable salts and solvates thereof.

In a further embodiment of the present invention, SOS1 inhibitors have been found to be particularly suitable for use in the treatment of pain when administered in combination with an anti NGF antibody.

The present invention provides a method of treating pain by administering a therapeutically effective amount of a compound of the present invention in combination with an anti-NGF antibody.

Tanezumab is an example of an anti-NGF antibody. Its a promising and highly efficacious pain therapy, but patients frequently suffer unpleasant side effects at dosage levels sufficient to provide pain relief.

The combination provides a cooperative level of efficacy, with the advantage that the anti-NGF antibody can be administered at a dosage levels sufficient to provide pain relief without reaching a level where an adverse event may be seen. In cooperative systems, two independent agents are able to show a level of activity equivalent to one of the agents at a much higher dose. Its surprising to find two agents combining to have such an effect.

It is possible to lower the dose of Tanezumab in humans and as a result reduce the propensity for side effects that limit use if of this class of drug. The combination of SOS1 inhibition with NGF blocking via monoclonal antibodies such as Tanezumab will deliver increased pain efficacy with reduced side effects when compared to the use of higher doses of Tanezumab alone.

Combinations of SOS1 inhibitors with NGF monoclonal antibodies, or other blockers/modulators of the NGF pathway, have the potential to deliver greater pain efficacy with reduced side effects leading to improved and enhanced treatment of pain in conditions such as osteoarthritis.

Accordingly, the present invention provides for the use of a compound of the present invention in combination with an anti NGF, wherein one or both components is administered at a sub-therapeutic dose for the treatment of pain.

The term sub therapeutic dose is used to describe to describe a dose lower than that at which the component shows efficacy as a monotherapy.

Other advantages for the combination include the potential for oral dosing instead of intravenous or sub-cutaneous dosing. The combination may also result in a lower cost of treatment and provide a lower risk of immunogenicity.

Particularly suitable anti NGF antibodies include Tanezumab, Fasinumab, Fulranumab and MEDI735.

Particularly suitable anti-NGF antibodies are Tanezumab and Fasinumab.

In a particularly preferred embodiment, the present invention provides for the use of compounds of the present invention in combination with a sub therapeutic dose of Tanezumab, for the treatment of pain. Optionally, both the compound of the present invention and Tanezumab are administered at a sub therapeutic dose.

Compounds which act as SOS1 inhibitors are known to be effective in the treatment in cancer. The present invention provides compounds of the present invention for the treatment of cancer. The compounds provide a method of treating cancer.

Suitable cancers may be selected from the group consisting of pancreatic cancer, lung cancer, colorectal cancer, cholangiocarcinoma, multiple myeloma, melanoma, uterine cancer, endometrial cancer, thyroid cancer, acute myeloid leukaemia, bladder cancer, urothelial cancer, gastric cancer, cervical cancer, head and neck squamous cell carcinoma, diffuse large B cell lymphoma, oesophageal cancer, chronic lymphocytic leukaemia, hepatocellular cancer, breast cancer, ovarian cancer, prostate cancer, glioblastoma, renal cancer and sarcomas.

In another aspect the suitable cancers are selected from the group consisting of pancreatic cancer, lung cancer (suitably non-small cell lung cancer (NSCLC)), cholangiocarcinoma and colorectal cancer.

In another aspect the present invention provides SOS1 inhibitors of formula (I) for conditions selected from RASopathy, suitably selected from the group consisting of Neurofibromatosis type 1 (N F1), Noonan Syndrome (NS), Noonan Syndrome with Multiple Lentigines (NSML) (also referred to as LEOPARD syndrome), Capillary Malformation-Arteriovenous Malformation Syndrome (CM-AVM), Costello Syndrome (CS), Cardio-Facio-Cutaneous Syndrome (CFC), Legius Syndrome (also known as NF1-like Syndrome) and Hereditary gingival fibromatosis.

In another aspect the disease/condition/cancer to be treated/prevented with the compounds of the present invention is a disease/condition/cancer defined as exhibiting one or more of the following molecular features:
1. KRAS alterations:
a. KRAS amplification (wt or mutant);
b. KRAS overexpression (wt or mutant);
c. KRAS mutation(s):
i. G12 mutations (e.g. G12C, G12V, G12S, G12A, G12V, G12R, G12F, G12D);
ii. G13 mutations (e.g. G13C, G13D, G13R, G13V, G13S, G13A) iii. T35 mutation (e.g. T35I);
iv. 136 mutation (e.g. 136L, 136M);
v. E49 mutation (e.g. E49K);
vi. Q61 mutation (e.g. Q61 H, Q61 R, Q61 P, Q61 E, Q61 K, Q61 L, Q61 K);
vii. K1 17 mutation (e.g. K1 17N);
viii. A146 mutation (e.g. A146T, A146V);
NRAS alterations:
a. NRAS amplification (wt or mutant);
b. NRAS overexpression (wt or mutant);
c. NRAS mutation(s):
G12 mutations (e.g. G12A, G12V, G12D, G12C, G12S, G12R); G13 mutation (e.g. G13V, G13D, G13R, G13S, G13C, G13A); Q61 mutation (e.g. Q61 K, Q61 L, Q61 H, Q61 P, Q61 R);
iv. A146 mutation (e.g. A146T, A146V);
HRAS alterations:
a. HRAS amplification (wt or mutant);
b. HRAS overexpression (wt or mutant);
c. HRAS mutation(s);
i. G12 mutation (e.g. G12C, G12V, G12S, G12A, G12V, G12R, G12F, G12D);
ii. G13 mutation (e.g. G13C, G13D, G13R, G13V, G13S, G13A);
iii. Q61 mutation (e.g. Q61 K, Q61 L, Q61 H, Q61 P, Q61 R);
EGFR alterations:
a. EGFR amplification (wt or mutant);
b. EGFR overexpression (wt or mutant);
c. EGFR mutation(s)
i. e.g. exon 20 insertion, exon 19 deletion (Del19), G719X (e.g. G719A, G719C, G719S), T790M, C797S, T854A, L858R, L861 Q, or any combination thereof;
ErbB2 (Her2) alterations:
a. ErbB2 amplification;
b. ErbB2 overexpression; c. ErbB2 mutation(s)
i. e.g. R678, G309, L755, D769, D769, V777, P780, V842, R896, c.2264_2278del (L755_T759del), c.2339_2340ins (G778_P780dup), S310;
6. c-MET alterations:
a. c-MET amplification;
b. c-MET overexpression;
c. c-MET mutation(s)
i. e.g. E168, N375, Q648, A887, E908, T1010, V1088, H1 1 12, R1 166, R1 188, Y1248,
Y1253, M1268, D1304, A1357, P1382;
7. AXL alterations:
a. AXL amplification;
b. AXL overexpression;
8. BCR-ABL alterations:
a. chromosomal rearrangements involving the ABL gene;
9. ALK alterations:
a. ALK amplification;
b. ALK overexpression;
c. ALK mutation(s)
i. e.g. 1 151 Tins, L1 152R, C1 156Y, F1 174L, L1 196M, L1 198F, G1202R, S1206Y, G1269A;
d. chromosomal rearrangements involving the ALK gene;
10. FGFR1 alterations:
a. FGFR1 amplification;
b. FGFR1 overexpression;
11. FGFR2 alterations:
a. FGFR2 amplification;
b. FGFR2 overexpression;
12. FGFR3 alterations:
a. FGFR3 amplification;
b. FGFR3 overexpression;
c. chromosomal rearrangement involving the FGFR3 gene; 13. NTRK1 alterations:
a. chromosomal rearrangements involving the NTRK1 gene;
14. NF1 alterations:
a. NF1 mutation(s);
15. RET alterations:
a. RET amplification;
b. RET overexpression;
c. chromosomal rearrangements involving the RET gene
16. ROS1 alterations:
a. ROS1 amplification;
b. ROS1 overexpression;
c. ROS1 mutation(s)
i. e.g. G2032R, D2033N, L2155S;
d. chromosomal rearrangements involving the ROS1 gene; 17. SOS1 alterations
a. SOS1 amplification;
b. SOS1 overexpression;
c. SOS1 mutation(s);
18. RAC1 alterations
a. RAC1 amplification;
b. RAC1 overexpression;
c. RAC1 mutation(s);
19. MDM2 alterations
a. MDM2 amplification
b. MDM2 overexpression
c. MDM2 amplification in combination with functional p53 d. MDM2 amplification in combination with wild-type p53
20. RAS wild-type
a. KRAS wild-type
a. HRAS wild-type
b. N RAS wild-type Particularly preferred, the cancer to be treated/prevented with the SOS1 inhibitor compound, SOS1 inhibitor compound for use, compound of formula (I), compound of formula (I) for use, use for preparing and method for the treatment and/or prevention as herein (above and below) defined is selected from the group consisting of:
   lung adenocarcinoma harboring a KRAS mutation selected from the group consisting of G12C, G12V, G12D and G12R;
   colorectal adenocarcinoma harboring a KRAS mutation selected from the group consisting of G12D, G12V, G12C, G12R and G13D; and
   pancreatic adenocarcinoma harboring a KRAS mutation selected from the group consisting of G12D, G12V, G12R, G12C and Q61 H.

According to another aspect of the invention there is provided the compounds of the present invention for separate, sequential or simultaneous use in a combination combined with a second pharmacologically active compound. Suitably the second pharmacologically active compound of the combination may include but is not limited to;
1. Inhibitors of EGFR and/or of Mutants Thereof
a. e.g. afatinib, erlotinib, gefitinib, lapatinib, cetuximab, panitumumab, osimertinib, olmutinib, EGF-816; b. preferred are afatinib, osimertinib and cetuximab;
c. most preferred is afatinib
2. inhibitors of ErbB2 (Her2) and/or of mutants thereof
a. e.g. afatinib, lapatinib, trastuzumab, pertuzumab;
b. preferred are afatinib and trastuzumab;
c. most preferred is trastuzumab;
3. inhibitors of ALK and/or of mutants thereof
a. e.g. crizotinib, alectinib, entrectinib, brigatinib;
b. preferred are crizotinib and alectinib;
c. most preferred is crizotinib;
4. inhibitors of MEK and/or of mutants thereof
a. e.g. trametinib, cobimetinib, binimetinib, selumetinib, refametinib;
b. preferred are trametinib and cobimetinib;
c. most preferred is trametinib;
5. inhibitors of KRAS G12C
a. e.g. ARS-853 (compound V-64 in WO 2014/152588), example 1-272 in WO 2016/044772;
6. inhibitors of BCR-ABL and/or of mutants thereof
a. e.g. imatinib, dasatinib, nilotinib;
b. preferred are imatinib and nilotinib;
c. most preferred is imatinib;
7. inhibitors of FGFR1 and/or FGFR2 and/or FGFR3 and/or of mutants thereof
a. e.g. nintedanib;
8. inhibitors of ROS1 and/or of mutants thereof
a. e.g. crizotinib, entrectinib, lorlatinib, ceritinib, merestinib;
b. preferred are crizotinib and entrectinib;
c. most preferred is crizotinib;
9. inhibitors of c-MET and/or of mutants thereof
10. inhibitors of AXL and/or of mutants thereof
11. inhibitors of NTRK1 and/or of mutants thereof 12. inhibitors of RET and/or of mutants
thereof
13. taxanes
a. e.g. paclitaxel, nab-paclitaxel, docetaxel;
b. preferred is paclitaxel;
14. platinum-containing compounds
a. e.g. cisplatin, carboplatin, oxaliplatin;
15. anf/-metabolites
a. e.g. 5-fluorouracil, capecitabine, floxuridine, cytarabine, gemcitabine, combination of trifluridine and tipiracil (=TAS102);
b. preferred is gemcitabine;
16. mitotic kinase inhibitors
a. e.g. CDK4/6 inhibitors
e.g. palbociclib, ribociclib, abemaciclib;
preferred are palbociclib and abemaciclib;
iii. most preferred is abemaciclib;
17. immunotherapeutic agents
a. e.g. immune checkpoint inhibitors
i. e.g. ani/-CTLA4 mAb, anf/-PD1 mAb, anti-PD-mAb, ani/-PD-L2 mAb, ani/-LAG3 mAb, ani/-TIM3 mAb;
ii. preferred are anf/-PD1 mAb;
iii. e.g. ipilimumab, nivolumab, pembrolizumab, atezolizumab, avelumab, durvalumab, pidilizumab, PDR-001 (BAPO49-Clone-E disclosed and used in WO 2017/019896);
iv. preferred are nivolumab, pembrolizumab and PDR-001;
v. most preferred is pembrolizumab;
18. anf/-angiogenic drugs
a. e.g. bevacizumab, nintedanib;
b. most preferred is bevacizumab;
19. topoisomerase inhibitors
a. e.g. irinotecan, liposomal irinotecan, topotecan;
b. most preferred is irinotecan; 20. inhibitors of A-Raf and/or B-Raf and/or C-Raf and/or of mutants thereof a. e.g. RAF-709 (=example 131 in WO 2014/151616), LY-3009120 (=example 1 in WO 2013/134243);
21. inhibitors of ERK and/or of mutants thereof
a. e.g. ulixertinib;
22. apoptose regulators
a. e.g. inhibitors of the interaction between p53 (suitably functional p53, most suitably wt p53) and MDM2 (“MDM2 inhibitors”);
i. e.g. HDM-201, NVP-CGM097, RG-71 12, MK-8242, RG-7388, SAR405838, AMG-232, DS-3032, RG-7775, APG-1 15;
ii. preferred are HDM-201, RG-7388 and AMG-232
b. e.g. PARP inhibitors;
c. e.g. MCL-1 inhibitors;
23. inhibitors of mTOR
a. e.g. rapamycin, temsirolimus, everolimus, ridaforolimus;
24. epigenetic regulators
a. e.g. BET inhibitors
i. e.g. JQ-1, GSK 525762, OTX 015 (=MK8628), CPI 0610, TEN-010 (=R06870810);
b. e.g. CDK9 inhibitors;
25. inhibitors of IGF1/2 and/or of IGF1-R
a. e.g. xentuzumab (antibody 60833 in WO 2010/066868), MEDI-573 (=dusigitumab);

Within this invention it is to be understood that the combinations, compositions, kits, methods, uses or compounds for use according to this invention may envisage the simultaneous, concurrent, sequential, successive, alternate or separate administration of the active ingredients or components.

Literature data indicates that integrin and Ab1-42 RAS/ERK signalling can be linked to tau hyperphosphorylation, focal adhesion development and other Alzheimer's disease neuropathology. Consistent with this, inhibitors of the pathway, such as MEK inhibitors, have been shown to ameliorate neuropathological related outcomes in cell disease models. In addition, in neurofibromatosis patients, a disease caused by excess signalling via SOS/RAS/ERK, the hazard ratio risk of AD is significantly increased. Hence, SOS1 inhibitors, that inhibit in the same pathway as MEK, could also provide benefit and offer additional efficacy and safety features both in Alzheimer's disease and other dementias. The present invention is also useful in the treatment of dementia, including:

Alzheimers, Mild cognitive impairment (MCI), Creutzfeldt-Jakob disease (CJD), Dementia with Lewy bodies (DLB), Vascular dementia, Alcohol-related brain damage (ARBD), Young-onset dementia, Frontotemporal dementia (FTD) & HIV-related cognitive impairment.

Alzheimers includes, Mild Alzheimer's A, Moderate Alzheimer's, Severe Alzheimer's, Inflammatory, Non-Inflammatory, Cortical, Early-Onset Alzheimer's & Late-Onset Alzheimer's′ Various literature data suggest that MAPK signaling pathways contribute to Parkinson's disease-related pathological processes, such as oxidative stress, neuro-inflammation, autophagy, and neuronal death. In addition, MEK inhibitors have demonstrated important neuroprotective properties upstream of the execution of apoptosis in dopaminergic neurons. It is expected that SOS1 inhibitors, that inhibit in the same pathway as MEK, could also have neuroprotective properties, with enhanced potency and safety versus MEK inhibitors.

The present invention is also useful in the treatment of parkinsonism also known as parkinsons disease. This includes, idiopathic Parkinson's, Vascular parkinsonism (also known as arteriosclerotic parkinsonism), drug-induced parkinsonism, multiple system atrophy, progressive supranucleur palsy, normal pressure hydrocephalus, tremors, including essential tremor & Wilsons disease.

The invention further provides a pharmaceutical formulation comprising a compound of formula I, as defined above, or a pharmaceutically acceptable salt or solvate thereof, and a pharmaceutically acceptable adjuvant, diluent or carrier. The pharmaceutical formulation may further comprise one or more additional active agents for the treatment of a disorder mentioned above.

The invention further provides a pharmaceutical kit comprising a compound of formula I, as defined above, or a pharmaceutically acceptable salt or solvate thereof, and one or more additional active agents, as a combined preparation for separate, simultaneous or sequential administration in the treatment of a disorder mentioned above.

The invention further provides a method of treatment of a disorder mentioned above in a mammal (especially a human), comprising administration of a therapeutically effective amount of a compound of formula I, as defined above, or a pharmaceutically acceptable salt or solvate thereof, to a mammal in need of such treatment.

Compounds of the invention intended for pharmaceutical use may be administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose.

They may be administered alone or in combination with one or more other compounds of the invention or in combination with one or more other drugs (or as any combination thereof). Generally, they will be administered as a formulation in association with one or more pharmaceutically acceptable excipients. The term ‘excipient’ is used herein to describe any ingredient other than the compound(s) of the invention. The choice of excipient will to a large extent depend on factors such as the particular mode of administration, the effect of the excipient on solubility and stability, and the nature of the dosage form.

Pharmaceutical compositions suitable for the delivery of compounds of the present invention and methods for their preparation will be readily apparent to those skilled in the art. Such compositions and methods for their preparation may be found, for example, in Remington's Pharmaceutical Sciences, 19th Edition (Mack Publishing Company, 1995).

### Oral Administration

The compounds of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films, ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986, by Liang and Chen (2001).

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, suitably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, suitably from 0.5 weight % to 3 weight % of the tablet.

Other possible ingredients include anti-oxidants, colourants, flavouring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in Pharmaceutical Dosage Forms: Tablets. Vol. 1, by H. Lieberman and L. Lachman (Marcel Dekker, New York, 1980).

Consumable oral films for human or veterinary use are typically pliable water-soluble or water-swellable thin film dosage forms which may be rapidly dissolving or mucoadhesive and typically comprise a compound of formula I, a film-forming polymer, a binder, a solvent, a humectant, a plasticiser, a stabiliser or emulsifier, a viscosity-modifying agent and a solvent. Some components of the formulation may perform more than one function.

The compound of the invention may be water-soluble or insoluble. A water-soluble compound typically comprises from 1 weight % to 80 weight %, more typically from 20 weight % to 50 weight %, of the solutes. Less soluble compounds may comprise a greater proportion of the composition, typically up to 88 weight % of the solutes. Alternatively, the compound of the invention may be in the form of multiparticulate beads.

The film-forming polymer may be selected from natural polysaccharides, proteins, or synthetic hydrocolloids and is typically present in the range 0.01 to 99 weight %, more typically in the range 30 to 80 weight %.

Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents.

Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Pharmaceutical Technology On-line. 25(2), 1-14, by Verma et al (2001). The use of chewing gum to achieve controlled release is described in WO 00/35298.

### Parenteral Administration

The compounds of the invention may also be administered directly into the blood stream, into muscle, or into an internal organ. Suitable means for parenteral administration include intravenous, intraarterial, intraperitoneal, intrathecal, intraventricular, intraurethral, intrastemal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (suitably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of the invention used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compounds of the invention may be formulated as a solid, semi-solid, or thixotropic liquid for administration as an implanted depot providing modified release of the active compound. Examples of such formulations include drug-coated stents and poly(dl-lactic-coglycolic)acid (PGLA) microspheres.

### Topical Administration

The compounds of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 0 (10), 955-958, by Finnin and Morgan (October 1999).

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release.

### Inhaled/Intranasal Administration

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (suitably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, suitably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of formula I, propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, PGLA. Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing from 1 to 10,000 μg of the compound of the invention. The overall daily dose will typically be in the range 1 μg to 10 mg which may be administered in a single dose or, more usually, as divided doses throughout the day.

### Rectal/Intravaginal Administration

The compounds of the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

### Ocular/Aural Administration

The compounds of the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

### Other Technologies

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

### Kit-of-Parts

Inasmuch as it may desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for coadministration of the compositions.

Thus the kit of the invention comprises two or more separate pharmaceutical compositions, at least one of which contains a compound of formula I in accordance with the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

### Dosage

For administration to human patients, the total daily dose of the compounds of the invention is typically in the range 0.5 mg to 3000 mg depending, of course, on the mode of administration. For example, oral administration may require a total daily dose of from 3 mg to 3000 mg, while an intravenous dose may only require from 0.5 mg to 500 mg. The total daily dose may be administered in single or divided doses and may, at the physician's discretion, fall outside of the typical range given herein.

These dosages are based on an average human subject having a weight of about 60 kg to 70 kg. The physician will readily be able to determine doses for subjects whose weight falls outside this range, such as infants and the elderly.

For the avoidance of doubt, references herein to “treatment” include references to curative, palliative and prophylactic treatment.

### EXPERIMENTAL

### Materials and Methods

The invention will now be illustrated by the following non-limiting examples in which, unless stated otherwise:
(i) temperatures are given in degrees Celsius (° C.); operations were carried out at room or ambient temperature, that is, at a temperature in the range of 18 to 25° C.;
(ii) final products had satisfactory proton and carbon nuclear magnetic resonance (NMR) spectra and/or mass spectral data;
(iii) yields are given for illustration only and are not necessarily those which can be obtained by diligent process development; preparations were repeated if more material was required;
(iv) when given, NMR data is in the form of delta values for major diagnostic protons, given in parts per million (ppm) relative to tetramethylsilane (TMS) as an internal standard, determined at 400 MHz using perdeuterio dimethyl sulfoxide (DMSO-d₆) as solvent unless otherwise indicated; the following abbreviations have been used: s, singlet; d, doublet; t, triplet; q, quartet; m, multiplet; bs, broad singlet, dd doublet of doublets;
(v) chemical symbols have their usual meanings; SI units and symbols are used.

List of Abbreviations used in the present invention are given below:
ACN Acetonitrile
AcOH Acetic acid
Aq Aqueous
BSA Bovine Serum Albumin
CS₂CO₃ Cesium carbonate
DCM Dichloromethane
DIPEAN, N-Diisopropylethylamine
DME Dimethoxy ethane
DMF Dimethylformamide
DMF-DMA N,N-Dimethylformamide dimethyl acetal
DMSO Dimethyl sulfoxide
DTT Dithiothreitol
EDA-GTP-DY-647P1 273′—O-(2-Aminoethyl-carbamoyl)-Guanosine-5′-triphosphate, labeled with DY-647PI,Triethylammonium salt
EtOAc Ethyl acetate
EtOH Ethanol
FRET Fluorescence resonance energy transfer
GDP Guanosine diphosphate
GEF Guanine nucleotide exchange factor
GTP Guanosine triphosphate
HATU (1-[Bis(dimethylamino)methylene]-1H-1,2,3-triazolo[4,5-b]pyridinium 3-oxide hexafluorophosphate
HEPES (4-(2-hydroxyethyl)-1-piperazineethanesulfonic acid)
HPLC High-performance liquid chromatography
HTRF Homogeneous Time Resolved Fluorescence
IPA Isopropanol
K₂CO₃ Potassium carbonate
KRAS Kirsten Rat Sarcoma
KO^{I}Bu Potassium tert-butoxide
LC Liquid chromatography
LiOH·H2O Lithium hydroxide monohydrate
MeOH Methanol
MeONa Sodium methoxide
MS Mass spectrometry
NaBH₄ Sodium borohydride
NaNC Sodium nitrite
NaIO₄ Sodium periodate
NH₄Cl Ammonium chloride
NiCH·6H2O Nickel(II) chloride hexahydrate
nM nanomolar
NMR Nuclear magnetic resonance spectroscopy
O/N Over night
Pd—C Palladium on carbon
Pd(PPhs)₄ Tetrakis(triphenylphosphine)palladium(0)
POCl₃ Phosphorus oxychloride
prep Preparative
TEA Triethylamine
THF Tetrahydrofuran
TFA Trifluoroacetic acid
TiCU Titanium tetrachloride
TLC Thin-layer chromatography

The following intermediates can be used to prepare compounds of the present invention.

### Example 1: (R)—N-(1-(3-(difluoromethyl)-2-fluorophenyl) ethyl)-6-methoxycinnolin-4-amine

### Preparation 1: 1-(2-amino-5-methoxyphenyl) ethan-1-one

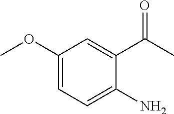

To a stirred solution 2-amino-5-methoxybenzonitrile (5.0 g, 0.034 mol) in dry THF dropwise at room temperature 3.0M CH₃MgBr in 2-Methyl THF (30 mL, 0.101 mol) was added dropwise. The resulting mixture was allowed to stir at room temperature for 24h. TLC (2:8 Ethyl acetate/Hexane) showed SM was consumed completely. Reaction mixture was quenched with sat. NH₄Cl solution, diluted with EtOAc and water. The combined organics were dried over anhydrous Na₂SO₄, filtered, and evaporated. The residue was purified by normal phase chromatography, eluting with (2:98) ethyl acetate/hexane to yield 1-(2-amino-5-methoxyphenyl) ethan-1-one (0.6 g, 11%) as a pale-yellow solid.

LCMS t_{R} (Waters Acquity UPLC with QDA mass Detector, Acidic, 4.0 min): 1.433 min, m/z=166.2 [M+H]⁺.

### Preparation 2: 6-methoxycinnolin-4-ol

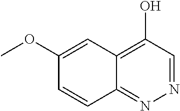

To a stirred solution 1-(2-amino-5-methoxyphenyl) ethan-1-one (0.3 g, 0.0018 mol) in conc. HCl (4.8 mL, 16V) at 0° C., Sodium nitrite (0.195 g, 0.0027 mol) in water was added dropwise. The resulting mixture was allowed to stir at room temperature for 6h. TLC (5:5 Ethyl acetate/Hexane) showed SM was consumed completely. Reaction mixture was quenched with sat. NaHCO₃ solution, concentrated under vacuum to afford crude. The residue was purified by normal phase chromatography, eluting with (45:55) ethyl acetate/hexane to yield 6-methoxycinnolin-4-ol (0.04 g, 13%) as a pale yellow solid.

LCMS t_{R} (Waters Acquity UPLC with QDA mass Detector, Acidic, 4.0 min): 1.275 min, m/z=177.2 [M+H]⁺.

### Preparation 3:4-bromo-6-methoxycinnoline

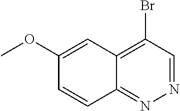

To a stirred solution 6-methoxycinnolin-4-ol (0.08 g, 0.00045 mol) in ACN, K₂CO₃ (0.188 g, 0.0014 mol) was added, followed by POBr₃ (0.39 g, 0.0014 mol). The resulting mixture was allowed to stir at 60° C. for 6h. TLC (5:5 Ethyl acetate/Hexane) showed SM was consumed completely. Reaction mixture was poured into ice-cold water, diluted with ethyl acetate. The combined organics were dried over anhydrous Na₂SO₄, filtered, and evaporated. The residue was purified by normal phase chromatography, eluting with (20:80) ethyl acetate/hexane to yield 4-bromo-6-methoxycinnoline (0.035 g, 33%) as a pale-yellow solid.

LCMS t_{R} (Waters Acquity UPLC with QDA mass Detector, Acidic, 4.0 min): 1.833 min, m/z=239.1 [M+H]⁺

### Example 1, Preparation 4: (R)—N-(1-(3-(difluoromethyl)-2-fluorophenyl) ethyl)-6-methoxycinnolin-4-amine

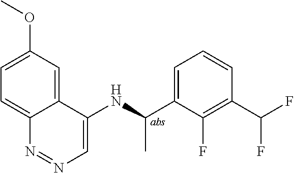 [SMILES: COc1ccc2nncc(N[C@H](C)c3cccc(C(F)F)c3F)c2c1]

To a stirred solution 4-bromo-6-methoxycinnoline (0.035 g, 0.00015 mol), (R)-1-(3-(difluoro methyl)-2-fluorophenyl) ethan-1-amine (0.042 g, 0.00022 mol), K₃PO₄ (0.095 g, 0.00045 mol), in toluene, purged with Nitrogen gas for 10 minutes followed by addition of Pd₂(dba)₃ (0.007 g, 0.0000075 mol) and Xant-phos (0.006 g, 0.000011 mol), again purged RM with nitrogen gas for 5 minutes. The resulting mixture was allowed to stir at 110° C. for 16h. TLC (6:4 Ethyl acetate/Hexane) showed SM was consumed completely. Reaction mixture was poured into ethyl acetate, diluted with brine solution. The combined organics were dried over anhydrous Na₂SO₄, filtered, and evaporated. The residue was purified by normal phase chromatography, eluting with (42:58) ethyl acetate/hexane to yield (R)—N-(1-(3-(difluoromethyl)-2-fluorophenyl) ethyl)-6-methoxycinnolin-4-amine (0.006 g, 12%) as a pale-yellow solid.

LCMS t_{R} (Waters Acquity UPLC with QDA mass Detector, Acidic, 4.0 min): 1.768 min, m/z=348.1 [M+H]⁺.

HPLC t_{R} (Waters Alliance e2695 with 2998 detector, Basic, 17.0 min): 4.713 min

1H NMR: (400 MHz, DMSO-d6): δ 8.23 (s, 1H), 8.06 (d, J=9.20 Hz, 1H), 7.81 (d, J=2.00 Hz, 1H), 7.62 (t, J=8.40 Hz, 1H), 7.52-7.54 (m, 2H), 7.43-7.43 (m, 1H), 7.25-7.29 (m, 2H), 5.26 (t, J=7.20 Hz, 1H), 4.00 (s, 3H), 1.70 (d, J=6.80 Hz, 3H).

### Example 2: (R)—N-(1-(3-(difluoromethyl)-2-fluorophenyl) ethyl)-6,7-dimethoxycinnolin-4-amine

### Preparation 1: 6,7-dimethoxycinnolin-4-ol

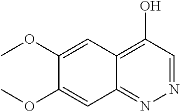

To a stirred solution of 1-(2-amino-4,5-dimethoxyphenyl) ethan-1-one (0.2 g, 2.42 mmol) in water (10V). NaNO₂ (0.1 g, 1.53 mmol) and Conc. HCl (3 ml) were added into reaction mixture. The resulting mixture was allowed to stir at RT for 2h. TLC (50% Ethyl acetate/Hexane) showed SM was consumed completely. The resulting solution was diluted with water (50 mL) and ethyl acetate (150 mL). The combined organics were dried over Na₂SO₄, filtered and evaporated. The crude was purified by manual Colum 50% Ethyl acetate in hexane yield 6,7-dimethoxycinnolin-4-ol (0.1 g, 47.34%) as a pale-yellow solid.

LCMS t_{R} (Waters Acquity UPLC with QDA mass Detector, Acidic, 4.0 min): 1.193 min, m/z=207. [M, M+2]⁺.

### Preparation 2: 4-bromo-6,7-dimethoxycinnoline

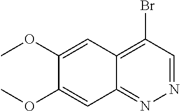

To a stirred solution of 6,7-dimethoxycinnolin-4-ol (0.5 g, 2.42 mmol) in ACN (5 ml). K₂CO₃ (0.66 g, 4.84 mmol) and POBr₃ (6.9 g, 121.2 mmol) added into reaction mixture at RT. The resulting mixture was allowed to stir at 80° C. for 16h. TLC (Neat Ethyl acetate) showed SM was consumed completely. The resulting solution was diluted with water (100 ml) and ethyl acetate (300 ml). The combined organics were dried over Na₂SO₄, filtered and evaporated. The Crude was purified by manual column 50% Ethyl acetate in hexane yield 4-bromo-6,7-dimethoxycinnoline (0.3 g, 45.98%) as an off-white solid.

LCMS t_{R} (Waters Acquity UPLC with QDA mass Detector, Acidic, 4.0 min): 1.820 min, m/z=269.1, 271.1 [M, M+2]⁺.

### Example 2, Preparation 3: (R)—N-(1-(3-(difluoromethyl)-2-fluorophenyl) ethyl)-6,7-dimethoxycinnolin-4-amine

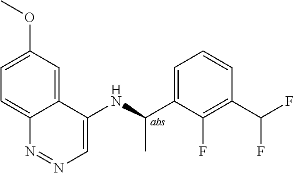 [SMILES: COc1ccc2nncc(N[C@H](C)c3cccc(C(F)F)c3F)c2c1]

To a stirred solution of 4-bromo-6,7-dimethoxycinnoline (0.2 g, 0.7 mmol) in Toluene (10V). (R)-1-(3-(difluoromethyl)-2-fluorophenyl) ethan-1-amine (0.281 g, 1.48 mmol) and K₃PO₄ (0.47 g, 2.22 mmol) were added to the above reaction mixture. The resulting mixture was allowed to stir at RT for 10 min. and N₂ gas was purged. Pd₂(dba)₃ (0.088 g, 0.073 mmol) and Xanth-phose (0.085 g, 0.14 mmol) were added to the above reaction mixture. The resulting mixture was allowed to stir at 100° C. for 16h. TLC (10% MeOH/MDC) showed SM was consumed completely. The resulting solution was diluted with water (50 mL) and ethyl acetate (150 mL). The combined organics were dried over Na2SO4, filtered and evaporated. The crude was purified by manual Colum 50% Ethyl acetate in hexane to yield (R)—N-(1-(3-(difluoromethyl)-2-fluorophenyl) ethyl)-6,7-dimethoxycinnolin-4-amine (0.07 g, 24.96%) as a pale brown solid.

LCMS t_{R} (Waters Acquit UPLC with QDA mass Detector, Acidic, 4.0 min): 1.809 min, m/z=378.2[M+H]⁺

HPLC t_{R} (Waters Alliance e2695 with 2998 detector, basic, 17.0 min): 5.62 min

1H NMR: (400 MHz, DMSO-d6): δ 8.17 (s, 1H), 7.76 (s, 1H), 7.60-7.53 (m, 2H), 7.44-7.42 (m, 2H), 7.32-7.15 (m, 2H), 5.26-5.22 (m, 1H), 4.01 (s, 3H), 3.95 (s, 3H), 1.69 (d, J=6.8 Hz, 3H).

### Example 3: 1N—(R)-1-(3-(difluoromethyl-2-fluorophenyl) ethyl-7-methoxy-6-(((S)-1-methylpyrrolidin-3-yl) methoxy) cinnolin-4-amine

### Preparation 1: (S)-4-bromo-7-methoxy-6-((1-methylpyrrolidin-3-yl) methoxy) cinnoline

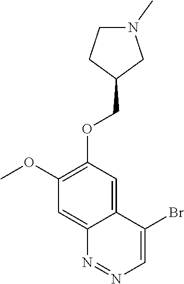 [SMILES: COc1cc2nncc(Br)c2cc1OC[C@H]1CCN(C)C1]

To a stirred solution of 4-bromo-6-fluoro-7-methoxycinnoline (0.1 g, 0.389 mmol) and (S)-(1-methylpyrrolidin-3-yl) methanol (0.044 g, 0.389 mmol) were dissolved in DMF (1 mL, 10V). LiHMDS (1M in THF) (0.58 mL, 0.58 mmol) was added dropwise to above solution. Then Suspension was stirred for 16 h at RT. TLC (0.5:9.5 MeOH/DCM) showed SM was consumed completely. The resulting solution was quenched with aq. NH₄Cl (100 mL) and extracted by ethyl acetate (50 mL). The combined organics were dried over Na₂SO₄, filtered, and evaporated. The residue was purified by normal phase chromatography, eluting with (10:90) MeOH/DCM to yield (S)-4-bromo-7-methoxy-6-((1-methylpyrrolidin-3-yl) methoxy) cinnoline (0.1 g, 72%) as an off-white sticky solid.

LCMS t_{R} (Waters Acquity UPLC with QDA mass Detector, Acidic, 4.0 min): 1.26 min, m/z=354.1 [M+2H]⁺.

### Example 3, Preparation 2: (R)-1-(3-(difluoromethyl)-2-fluorophenyl) ethyl)-7-methoxy-6-(((S)-1-methylpyrrolidin-3-yl) methoxy) cinnolin-4-amine

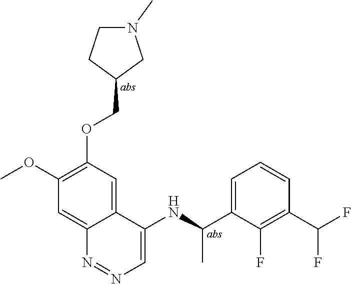 [SMILES: COc1cc2nncc(N[C@H](C)c3cccc(C(F)F)c3F)c2cc1OC[C@H]1CCN(C)C1]

To a stirred solution of (S)-4-bromo-7-methoxy-6-((1-methylpyrrolidin-3-yl) methoxy) cinnoline (0.1 g, 0.284 mmol), Scaffold-A (0.08 g, 0.427 mmol) and Potassium phosphate (0.18 g, 0.056 mmol) were dissolved in Toluene (2 mL, 20V). Pd₂(dba)s (0.025 g, 0.0284 mmol) and Xant-phos (0.032 g, 0.056 mmol) were added to the mixture. The resulting mixture was allowed to stir at 120° C. for 3h. TLC (2:8/MeOH: DCM) showed SM was consumed completely. The resulting solution was diluted with water (100 mL) and extracted with ethyl acetate (100 mL). The organics were dried and concentrated under vacuum. The residue was purified by reverse phase chromatography, eluting with 40-45% acetonitrile in water to yield (R)-1-(3-(difluoromethyl)-2-fluorophenyl) ethyl)-7-methoxy-6-(((S)-1-methylpyrrolidin-3-yl) methoxy) cinnolin-4-amine (0.006 g, 4.5%) as a fluffy white solid.

LCMS t_{R} (Waters Acquity UPLC with QDA mass Detector, Acidic, 4.0 min): 6.39 min, m/z=461.3 [M+H]⁺.

HPLC t_{R} (Waters Alliance e2695 with 2998 detector, Basic, 17.0 min): 4.58 min

1H NMR (400 MHz, DMSO): δ 8.16 (s, 1H), 7.74 (s, 1H), 7.60 (t, J=8.40 Hz, 1H), 7.55 (t, J=6.40 Hz, 1H), 7.45 (s, 1H), 7.38-7.42 (m, 1H), 7.29-7.32 (m, 2H), 5.22-5.27 (m, 1H), 4.05-4.15 (m, 2H), 3.95 (s, 3H), 2.55-2.71 (m, 3H), 2.30 (s, 3H), 1.91-2.07 (m, 2H), 1.69 (d, J=6.80 Hz, 3H), 1.60-1.63 (m, 2H).

### Example 4: 1-((S)-3-((4-(((R)-1-(3-(difluoromethyl)-2-fluorophenyl) ethyl) amino)-7-methoxycinnolin-6-yl) oxy) pyrrolidin-1-yl) ethan-1-one

### Preparation 1: (S)-1-(3-((4-bromo-7-methoxycinnolin-6-yl) oxy) pyrrolidin-1-yl) ethan-1-one

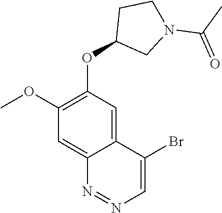 [SMILES: COc1cc2nncc(Br)c2cc1O[C@H]1CCN(C(C)=O)C1]

To a stirred solution of 4-bromo-6-fluoro-7-methoxycinnoline (0.1 g, 0.389 mmol) and (S)-1-(3-hydroxypyrrolidin-1-yl) ethan-1-one (0.050 g, 0.389 mmol) were dissolved in DMF (1 mL, 10V). LiHMDS (1M in THF) (0.58 mL, 0.58 mmol) was added dropwise to above solution. Then Suspension was stirred for 16 h at RT. TLC (1:9 MeOH/DCM) showed SM was consumed completely. The resulting solution was quenched with aq. NH₄Cl (100 mL) and extracted by ethyl acetate (50 mL). The combined organics were dried over Na2SO4, filtered, and evaporated. The residue was purified by normal phase chromatography, eluting with (10:90) MeOH/DCM to yield (S)-1-(3-((4-bromo-7-methoxycinnolin-6-yl) oxy) pyrrolidin-1-yl) ethan-1-one (0.1 g, 70%) as a brown sticky liquid.

LCMS t_{R} (Waters Acquity UPLC with QDA mass Detector, Acidic, 4.0 min): 1.51 min, m/z=368.1 [M+2H]⁺.

### Example 4, Preparation 2: 1-((S)-3-((4-(((R)-1-(3-(difluoromethyl)-2-fluorophenyl) ethyl) amino)-7-methoxycinnolin-6-yl) oxy) pyrrolidin-1-yl) ethan-1-one

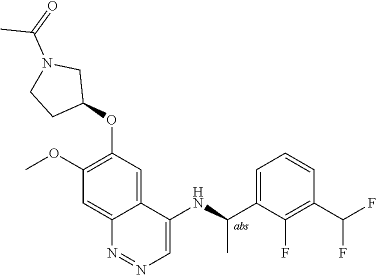 [SMILES: COc1cc2nncc(N[C@H](C)c3cccc(C(F)F)c3F)c2cc1O[C@H]1CCN(C(C)=O)C1]

To a stirred solution of (S)-4-bromo-7-methoxy-6-((1-methylpyrrolidin-3-yl) methoxy) cinnoline (0.09 g, 0.245 mmol), Scaffold-A (0.07 g, 0.368 mmol) and Sodium tert-butoxide (0.07 g, 0.056 mmol) were dissolved in Toluene (2 mL, 20V). Pd₂(dba)₃ (0.022 g, 0.024 mmol) and Xant-phos (0.028 g, 0.049 mmol) were added to the mixture. The resulting mixture was allowed to stir at 120° C. for 16h. TLC (1:9/MeOH: DCM) showed SM was consumed completely. The resulting solution was diluted with water (100 mL) and extracted with ethyl acetate (100 mL). The organics were dried and concentrated under vacuum. The residue was purified by reverse phase chromatography, eluting with 25-30% acetonitrile in water to yield 1-((S)-3-((4-(((R)-1-(3-(difluoromethyl)-2-fluorophenyl) ethyl) amino)-7-methoxycinnolin-6-yl) oxy) pyrrolidin-1-yl) ethan-1-one (0.013 g, 11%) as a white solid.

LCMS t_{R} (Waters Acquity UPLC with QDA mass Detector, Acidic, 4.0 min): 1.83 min, m/z=474.9 [M+H]⁺.

HPLC t_{R} (Waters Alliance e2695 with 2998 detector, Basic, 17.0 min): 4.85 min

1H NMR (400 MHz, DMSO): δ 8.18 (s, 1H), 7.77 (s, 1H), 7.54-7.62 (m, 2H), 7.47-7.48 (m, 1H), 7.41-7.42 (m, 1H), 7.28-7.32 (m, 2H), 5.24-5.29 (m, 2H), 3.95 (s, 3H), 3.58-3.71 (m, 4H), 3.33-3.35 (m, 2H), 1.98-2.00 (m, 3H), 1.70 (d, J=6.8 Hz, 3H).

### Example 5: (R)-4-((4-((1-(3-(difluoromethyl)-2-fluorophenyl)ethyl)amino)-7-methoxycinnolin-6-yl)oxy)tetrahydro-2H-thiopyran 1,1-dioxide

### Preparation 1: 4-((4-bromo-7-methoxycinnolin-6-yl) oxy) tetrahydro-2H-thiopyran 1,1-dioxide

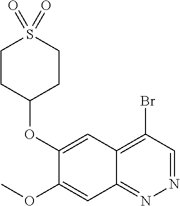 [SMILES: COc1cc2nncc(Br)c2cc1OC1CCS(=O)(=O)CC1]

To a stirred solution of 4-bromo-6-fluoro-7-methoxycinnoline (0.08 g, 0.311 mmol, 1.0 eq) and 4-hydroxytetrahydro-2H-thiopyran 1,1-dioxide (0.046 g, 0.311 mmol, 1.0 eq) in DMF (1 mL). added Lithium bis(trimethylsilyl)amide solution (1.0 M in THF) (0.46 ml, 0.466 mmol, 1.5 eq) The reaction mixture was stirred under RT for 16 hours. TLC (8:2 Ethyl acetate/Hexane) showed SM was consumed completely. The residue was purified by trituration (diethyl ether) to yield 4-((4-bromo-7-methoxycinnolin-6-yl) oxy) tetrahydro-2H-thiopyran 1,1-dioxide (0.07 g, 58.08 10%) as a brown solid.

LCMS t_{R} (Waters Acquity UPLC with QDA mass Detector, Acidic, 4.0 min): 1.785 min, m/z=396.7[M+H]⁺.

1H NMR: (400 MHz, DMSO): δ 9.43 (s, 1H), 7.85 (s, 1H), 7.39 (s, 1H), 5.17-5.18 (m, 1H), 4.08 (s, 3H), 3.29 (t, J=4.40 Hz, 2H), 3.21-3.22 (m, 2H), 2.34 (s, 4H).

### Example 5. Preparation 2: (R)-4-((4-((1-(3-(difluoromethyl)-2-fluorophenyl)ethyl)amino)-7-methoxycinnolin-6-yl) oxy)tetrahydro-2H-thiopyran 1,1-dioxide

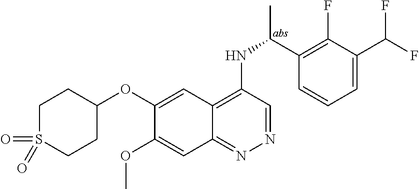 [SMILES: COc1cc2nncc(N[C@H](C)c3cccc(C(F)F)c3F)c2cc1OC1CCS(=O)(=O)CC1]

To a stirred solution of 4-((4-bromo-7-methoxycinnolin-6-yl) oxy) tetrahydro-2H-thiopyran 1,1-dioxide (0.065 g, 0.167 mmol), (R)-1-(3-(difluoromethyl)-2-fluorophenyl) ethan-1-amine (0.047 g, 0.251 mmol) and Tri potassium phosphate (0.106 g, 0.503 mmol) in Toluene (10V). The reaction mixture was degassed with nitrogen for 20 min. then added Pd₂(dba)s (0.015 g, 0.016 mmol) and Xant-phos (0.019 g, 0.033 mmol) was added to the above mixture. The resulting mixture was allowed to stir at 120° C. for 24h. TLC (10% Methanol/Dichloromethane) showed SM was consumed completely. The resulting solution was diluted with water (10 mL) and ethyl acetate (3×10 mL). The combined organics were dried over Na₂SO₄, filtered, and evaporated. The residue was purified by normal phase chromatography, eluting with (10:90) Methanol/Dichloromethane to yield (R)-4-((4-((1-(3-(difluoromethyl)-2-fluorophenyl) ethyl) amino)-7-methoxycinnolin-6-yl) oxy) tetrahydro-2H-thiopyran 1,1-dioxide (0.0098 g, 11.82%) as a Light-Yellow solid.

LCMS t_{R} (Waters Acquity UPLC with QDA mass Detector, Acidic, 4.0 min): 1.881 min, m/z=495.9 [M+H]⁺.

HPLC t_{R} (Waters Alliance e2695 with 2998 detector, Basic, 17.0 min): 4.956 min

1H NMR (400 MHz, DMSO-d6): δ 8.18 (s, 1H), 7.98 (s, 1H), 7.61-7.64 (m, 1H), 7.51-7.58 (m, 2H), 7.40 (t, J=7.20 Hz, 1H), 7.30 (t, J=8.00 Hz, 2H), 5.24-5.20 (m, 1H), 4.98 (bs, 1H), 3.99 (s, 3H), 3.26-3.27 (m, 2H), 3.18-3.21 (m, 2H), 2.40-2.49 (m, 2H), 2.30-2.39 (m, 2H), 1.69 (d, J=6.8 Hz, 3H).

### Example 6: (R)-1-(3-((4-((1-(3-(difluoromethyl)-2-fluorophenyl)ethyl)amino)-7-methoxycinnolin-6-yl)oxy)azetidin-1-yl)ethen-1-one

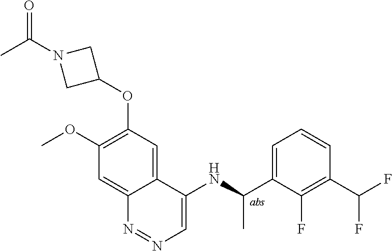 [SMILES: COc1cc2nncc(N[C@H](C)c3cccc(C(F)F)c3F)c2cc1OC1CN(C(C)=O)C1]

To a stirred solution of (R)—N-(1-(3-(difluoromethyl)-2-fluorophenyl) ethyl)-6-fluoro-7-methoxycinnolin-4-amine (0.09 g, 0.246 mmol) and 1-(3-hydroxyazetidin-1-yl) ethan-1-one (0.14 g, 1.232 mmol) were dissolved in DMF (1 mL, 10V). Potassium carbonate (0.23 g, 1.72 mmol) was added to the mixture. Then Suspension was stirred for 48 h at 120° C. TLC (9:1, DCM: MeOH) showed SM was consumed completely. The resulting solution was diluted with water (100 mL) and extracted with ethyl acetate (100 mL). The organics were dried and concentrated under vacuum. The residue was purified by reverse phase chromatography, eluting with 45-50% acetonitrile in water to yield (R)-1-(3-((4-((1-(3-(difluoromethyl)-2-fluorophenyl)ethyl)amino)-7-methoxycinnolin-6-yl)oxy)azetidin-1-yl)ethan-1-one (0.023 g, 21%) as an off-white solid.

LCMS t_{R} (Waters Acquity UPLC with QDA mass Detector, Acidic, 4.0 min): 1.66 min, m/z=461.4[M+H]⁺.

HPLC t_{R} (Waters Alliance e2695 with 2998 detector, Basic, 17.0 min): 5.30 min

1H NMR: (400 MHz, DMSO): δ 8.19 (s, 1H), 7.54-7.62 (m, 2H), 7.49-7.50 (m, 2H), 7.37-7.42 (m, 1H), 7.28-7.33 (m, 2H), 5.23-5.28 (m, 2H), 4.70 (t, J=7.20 Hz, 1H), 4.45-4.52 (m, 1H), 4.18-4.25 (m, 1H), 3.98 (s, 3H), 3.83-3.90 (m, 1H), 1.85 (s, 3H), 1.70 (d, J=6.80 Hz, 3H).

### Example-7: (R—N-(1(3-(difluoromethyl)-2-fluorophenylethyl)-7-methoxy-6-morpholinocinnolin-4-amine

### Preparation 1: 4-(4-bromo-7-methoxycinnolin-6-yl) morpholine

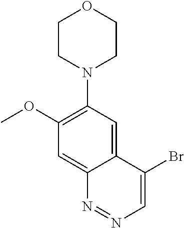 [SMILES: COc1cc2nncc(Br)c2cc1N1CCOCC1]

To a stirred solution of 4-bromo-6-fluoro-7-methoxycinnoline (0.09 g, 0.389 mmol) and morpholine (0.039 g, 0.45 mmol) were dissolved in DMF (1 mL, 10V). LiHMDS (1M in THF) (0.52 mL, 0.52 mmol) was added dropwise to above solution. Then Suspension was stirred for 16 h at RT. TLC (1:9 MeOH/DCM) showed SM was consumed completely. The resulting solution was quenched with aq. NH₄Cl (100 mL) and extracted by ethyl acetate (50 mL). The combined organics were dried over Na₂SO₄, filtered, and evaporated. The residue was purified by normal phase chromatography, eluting with (10:90) MeOH/DCM to yield (4-(4-bromo-7-methoxycinnolin-6-yl) morpholine (0.09 g, 79.3%) as a brown sticky liquid.

LCMS t_{R} (Waters Acquity UPLC with QDA mass Detector, Acidic, 4.0 min): 1.91 min, m/z=325.7 [M+2H]⁺.

### Example 7, Preparation 2: (R)—N-(1-(3-(difluoromethyl)-2-fluorophenyl)ethyl)-7-methoxy-6-morpholinocinnolin-4-amine

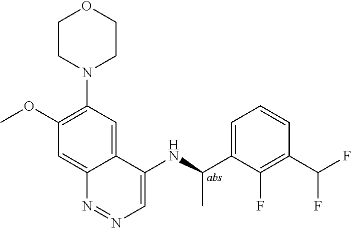 [SMILES: COc1cc2nncc(N[C@H](C)c3cccc(C(F)F)c3F)c2cc1N1CCOCC1]

To a stirred solution of (4-(4-bromo-7-methoxycinnolin-6-yl) morpholine (0.09 g, 0.26 mmol), Scaffold-A (0.074 g, 0.393 mmol) and Sodium tert-butoxide (0.075 g, 0.078 mmol) were dissolved in Toluene (1.8 mL, 20V). Pd₂(dba)s (0.024 g, 0.026 mmol) and Xant-phos (0.030 g, 0.052 mmol) were added to the mixture. The resulting mixture was allowed to stir at 120° C. for 16 h. TLC (0.5:9.5/MeOH: DCM) showed SM was consumed completely. The resulting solution was diluted with water (10 mL) and extracted with ethyl acetate (10 mL). The organics were dried and concentrated under vacuum. The residue was purified by Prep-HPLC purification to yield (R)—N-(1-(3-(difluoromethyl)-2-fluorophenyl)ethyl)-7-methoxy-6-morpholinocinnolin-4-amine (0.018 g, 14.99%) as a Yellow thick gum.

LCMS t_{R} (Waters Acquity UPLC with QDA mass Detector, Acidic, 4.0 min): 1.93 min, m/z=433 [M+H]⁺.

HPLC t_{R} (Waters Alliance e2695 with 2998 detector, Basic, 17.0 min): 4.07 min

1H NMR (400 MHz, DMSO): δ 9.32 (s, 1H), 8.48 (s, 1H), 7.83 (s, 1H), 7.74 (t, J=6.80 Hz, 1H), 7.63 (t, J=6.80 Hz, 1H), 7.40 (t, J=8.00 Hz, 1H), 7.25 (d, J=9.20 Hz, 2H), 5.71 (d, J=13.60 Hz, 1H), 4.04 (s, 3H), 3.80-3.82 (m, 4H), 3.22-3.23 (m, 4H), 1.77 (d, J=6.80 Hz, 3H).

### Example-8: 6-(6-oxa-3-azabicyclo [3.1.1] heptan-3-yl)-N—((R)-1-(3-(difluoromethyl)-2 fluorophenyl) ethyl-7-methoxycinnolin-4-amine

### Preparation 1: 3-(4-bromo-7-methoxycinnolin-6-yl)-6-oxa-3-azabicyclo [3.1.1] heptane

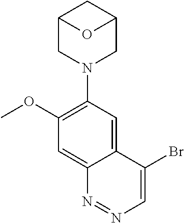 [SMILES: COc1cc2nncc(Br)c2cc1N1CC2CC(C1)O2]

To a stirred solution of 4-bromo-6-fluoro-7-methoxycinnoline (0.1 g, 0.38 mmol) and 6-oxa-3-azabicyclo [3.1.1]heptane (0.057 g, 0.42 mmol) were dissolved in DMF (1 mL, 10V). LIHMDS (1M in THF) (0.57 mL, 0.57 mmol) was added dropwise to above solution. Then Suspension was stirred for 16 h at RT. TLC (1:9 MeOH/DCM) showed SM was consumed completely. The resulting solution was quenched with aq. NH₄Cl (100 mL) and extracted by ethyl acetate (50 mL). The combined organics were dried over Na₂SO₄, filtered, and evaporated. The residue was purified by normal phase chromatography, eluting with (10.90) MeOH/DCM to yield 3-(4-bromo-7-methoxycinnolin-6-yl)-6-oxa-3-azabicyclo [3.1.1]heptane (0.1 g, 76.4%) as a brown solid.

LCMS t_{R} (Waters Acquity UPLC with QDA mass Detector, Acidic, 4.0 min): 1.80 min, m/z=337.5 [M+2H]⁺

### Example 8, Preparation 2: 6-(6-oxa-3-azabicyclo [3.1.1] heptan-3-yl)-N—((R)-1-(3-(difluoromethyl)-2-fluorophenyl) ethyl)-7-methoxycinnolin-4-amine

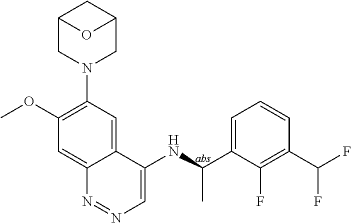 [SMILES: COc1cc2nncc(N[C@H](C)c3cccc(C(F)F)c3F)c2cc1N1CC2CC(C1)O2]

To a stirred solution of 3-(4-bromo-7-methoxycinnolin-6-yl)-6-oxa-3-azabicyclo [3.1.1]heptane (0.1 g, 0.29 mmol), Scaffold-A (0.067 g, 0.35 mmol) and K₃PO₄ (0.184 g, 0.87 mmol) were dissolved in Toluene (1.0 mL, 10V). Pd₂(dba)s (0.013 g, 0.014 mmol) and Xant-phos (0.017 g, 0.029 mmol) were added to the mixture. The resulting mixture was allowed to stir at 120° C. for 2 h. TLC (1:9/MeOH: DCM) showed SM was consumed completely. The resulting solution was diluted with water (10 mL) and extracted with ethyl acetate (10 mL). The organics were dried and concentrated under vacuum. The residue was purified by revers phase chromatography to yield (6-(6-oxa-3-azabicyclo [3.1.1]heptan-3-yl)-N—((R)-1-(3-(difluoromethyl)-2-fluorophenyl) ethyl)-7-methoxycinnolin-4-amine (0.018 g, 13.62%) as a Yellow Solid.

LCMS t_{R} (Waters Acquity UPLC with QDA mass Detector, Acidic, 4.0 min): 1.96 min, m/z=445 [M+H]⁺.

HPLC t_{R} (Waters Alliance e2695 with 2998 detector, Basic, 16.0 min): 6.29 min

1H NMR (400 MHz, DMSO): δ 8.12 (s, 1H), 7.54-7.62 (m, 3H), 7.45 (s, 1H), 7.38 (d, J=7.20 Hz, 1H), 7.29-7.33 (m, 2H), 5.22-5.27 (m, 1H), 4.69-4.70 (m, 2H), 4.05 (d, J=1.60 Hz, 1H), 4.02 (s, 3H), 3.91 (d, J=10.80 Hz, 1H), 3.72 (d, J=12.00 Hz, 1H), 3.61 (d, J=12.00 Hz, 1H), 3.11-3.13 (m, 1H), 2.26 (d, J=8.40 Hz, 1H), 1.69 (d, J=6.80 Hz, 3H).

### Example 9: N—((R)-1-(3-(difluoromethyl)-2-fluorophenyl) ethyl)-7-methoxy-6-(((S)-pyrrolidin-3-yl) oxy) cinnolin-4-amine

Preparation 1: tert-butyl (S)-3-((4-bromo-7-methoxycinnoline-6-yl) oxy) pyrrolidine-1-carboxylate

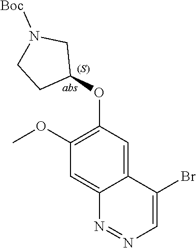

To a stirred solution of 4-bromo-6-fluoro-7-methoxycinnoline (0.62 g, 2.412 mmol) and tert-butyl (S)-3-hydroxypyrrolidine-1-carboxylate (0.67 g, 3.618 mmol) were dissolved in DMF (1 mL, 10V). LiHMDS (1M in THF) (6.03 mL, 6.031 mmol) was added dropwise to above solution. Then Suspension was stirred for 2 h at RT. TLC (1:9 MeOH/DCM) showed SM was consumed completely. The resulting solution was quenched with aq. NH₄Cl (100 mL) and extracted by ethyl acetate (100 mL). The combined organics were dried over Na2SO4, filtered, and evaporated. The residue was purified by normal phase chromatography, eluting with (10:90) MeOH/DCM to yield tert-butyl (S)-3-((4-bromo-7-methoxycinnolin-6-yl) oxy) pyrrolidine-1-carboxylate (0.85 g, 100%) as a brown solid.

LCMS t_{R} (Waters Acquity UPLC with QDA mass Detector, Acidic, 4.0 min): 2.21 min, m/z=425.7 [M+2H]⁺.

### Preparation 2: tert-butyl (S)-3-((4-(((R)-1-(3-(difluoromethyl)-2-fluorophenyl)ethyl)amino)-7-methoxy cinnolin-6-yl)oxy)pyrrolidine-1-carboxylate

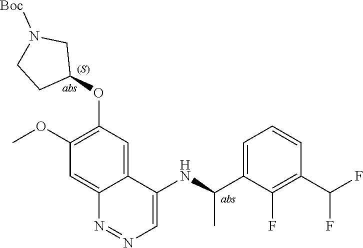

To a stirred solution of tert-butyl (S)-3-((4-bromo-7-methoxycinnolin-6-yl) oxy) pyrrolidine-1-carboxylate (0.15 g, 0.353 mmol), (R)-1-(3-(difluoromethyl)-2-fluorophenyl) ethan-1-amine (0.10 g, 0.53 mmol) and K₃PO₄ (0.25 g, 1.06 mmol) were dissolved in Toluene (3 mL, 20V). Pd₂(ba)₃ (0.032 g, 0.035 mmol) and Xant-phos (0.041 g, 0.070 mmol) were added to the mixture. The resulting mixture was allowed to stir at 120° C. for 2h. TLC (1:9/MeOH: DCM) showed SM was consumed completely. The resulting solution was diluted with water (100 mL) and extracted with ethyl acetate (100 mL). The organics were dried and concentrated under vacuum. The residue was purified by normal phase chromatography, eluting with 5% methanol in dichloromethane to yield tert-butyl (S)-3-((4-(((R)-1-(3-(difluoromethyl)-2-fluorophenyl) ethyl) amino)-7-methoxy cinnolin-6-yl)oxy)pyrrolidine-1-carboxylat e (0.075 g, 26%) as a Brown solid.

LCMS t_{R} (Waters Acquity UPLC with QDA mass Detector, Acidic, 4.0 min): 1.95 min, m/z=533.3 [M+H]⁺.

### Preparation Example 9: N—((R)-1-(3-(difluoromethyl)-2-fluorophenyl) ethyl)-7-methoxy-6-(((S)-pyrrolidin-3-yl) oxy) cinnolin-4-amine

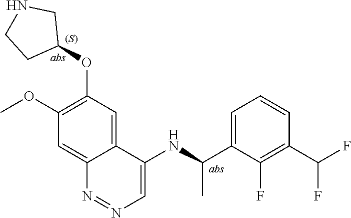

To a solution of tert-butyl (S)-3-((4-(((R)-1-(3-(difluoromethyl)-2-fluorophenyl) ethyl) amino)-7-methoxycinnolin-6-yl)oxy)pyrrolidine-1-carboxylate (0.2 g, 0.33 mmol) was dissolved in Dichloromethane (0.75 mL, 10V). Trifluoracetic acid (0.22 mL, 3V) was added to the above solution at 0° C. The suspension was stirred at room temperature for 2h. TLC (10:90 DCM/MeOH) showed SM was consumed completely. The resulting mixture was concentrated under vacuum. The crude material was purified using PREP-HPLC purification to yield N—((R)-1-(3-(difluoromethyl)-2-fluorophenyl)ethyl)-7-methoxy-6-(((S)-pyrrolidin-3-yl)oxy)cinnolin-4-amine (0.028 g, 45%) as an off-white solid.

LCMS t_{R} (Waters Acquity UPLC with QDA mass Detector, Acidic, 4.0 min): 1.74 min, m/z=433.4 [M+H]⁺.

HPLC t_{R} (Waters Alliance e2695 with 2998 detector, Basic, 17.0 min): 3.42 min

1H NMR (400 MHz, DMSO): δ 8.17 (s, 1H), 7.71 (s, 1H), 7.57-7.62 (m, 2H), 7.39-7.47 (m, 2H), 7.15-7.33 (m, 2H), 6.57 (s, 1H), 5.21-5.28 (m, 2H), 3.94 (s, 3H), 3.21-3.25 (m, 1H), 2.96-3.01 (m, 3H), 2.20-2.25 (m, 1H), 1.90-1.94 (m, 1H), 1.69 (d, J=6.4 Hz, 3H).

### Example 10: N—((R)-1-(3-(difluoromethyl)-2-fluorophenyl)ethyl)-7-methoxy-6-(((S)-tetrahydrofuran-3-yl)oxy)cinnolin-4-amine

### Preparation 1: N—((R)-1-(3-(difluoromethyl)-2-fluorophenyl)ethyl)-7-methoxy-6-(((S)-tetrahydrofuran-3-yl)oxy)cinnolin-4-amine

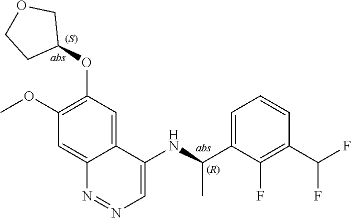

To a stirred solution of (R)—N-(1-(3-(difluoromethyl)-2-fluorophenyl)ethyl)-6-fluoro-7-methoxycinnolin-4-amine (0.08 g, 0.219 mmol, 1.0 eq) and in DMF (1 mL). added Caesium carbonate (0.214 g, 0.657 mmol, 3.0 eq) and (S)-tetrahydrofuran-3-ol (0.038 g, 0.438 mmol, 1.0 eq) was added into stirred solution. The reaction mixture was stirred under 110° C. for 6 hours. TLC (5:9.5 Methanol/Dichloromethane) showed SM was consumed completely. The residue was purified by reverse phase chromatography, eluting with (60:40) Acetonitrile/Water to yield N—((R)-1-(3-(difluoromethyl)-2-fluorophenyl)ethyl)-7-methoxy-6-(((S)-tetrahydrofuran-3-yl)oxy)cinnolin-4-amine(0.007 g, 7.48%) as a off-white solid.

LCMS t_{R} (Waters Acquity UPLC with QDA mass Detector, Acidic, 4.0 min): 1.779 min, m/z=434.2[M+H]⁺.

HPLC t_{R} (Waters Alliance e2695 with 2998 detector, Basic, 17.0 min): 5.67 min.

1H NMR (400 MHz, DMSO): δ 8.18 (s, 1H), 7.73-72 (m, 1H), 7.53-7.61 (m, 2H), 7.46 (s, 1H), 7.40 (t, J=6.80 Hz, 1H), 7.30 (t, J=8.80 Hz, 2H), 5.33 (bs, 1H), 5.23-5.28 (m, 1H), 4.04-4.05 (m, 1H), 3.95 (s, 3H), 3.81-3.83 (m, 3H), 2.42-2.44 (m, 1H), 2.05-2.11 (m, 1H), 1.69 (d, J=6.80 Hz, 3H).

Further compounds were made by the synthetic routes described herein:

### Example 11—R)-1-(4-((4-((1-(3-(difluoromethyl)-2-fluorophenyl)ethyl)amino)-7-methoxycinnolin-6-yl)oxy)piperidin-1-yl)ethan-1-one

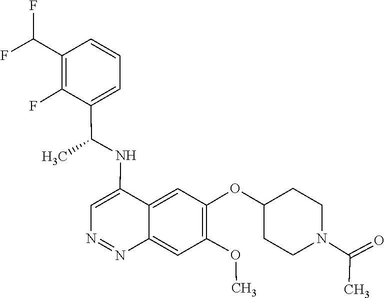 [SMILES: COc1cc2nncc(N[C@H](C)c3cccc(C(F)F)c3F)c2cc1OC1CCN(C(C)=O)CC1]

1H nmr: δ 8.20 (d, J=6.8 Hz, 1H), 8.03 (s, 1H), 7.59-7.52 (m, 2H), 7.42-7.14 (m, 3H), 7.32-7.28 (m, 2H), 5.21-5.18 (m, 1H), 4.36-4.34 (m, 1H), 3.94 (s, 3H), 3.74-3.69 (m, 2H), 3.60-3.54 (m, 2H), 2.05 (s, 3H), 1.97-1.94 (m, 2H), 1.80-1.75 (m, 2H), 121.69 (d, J=6.8 Hz, 3H).

### Example 12—(R)—N-(1-(3-(difluoromethyl)-2-fluorophenyl) ethyl)-7-methoxy-6-(piperidin-4-yloxy) cinnolin-4-amine

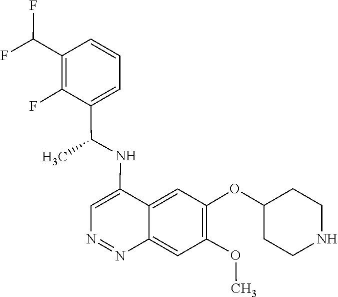 [SMILES: COc1cc2nncc(N[C@H](C)c3cccc(C(F)F)c3F)c2cc1OC1CCNCC1]

1H nmr: 6 8.62 (s, 2H), 8.50 (s, 1H), 8.15 (s, 1H), 7.74 (t, J=7.20 Hz, 1H), 7.63 (d, J=7.20 Hz, 1H), 7.35-7.40 (m, 2H), 7.26 (s, 1H), 5.69-5.73 (m, 1H), 4.96 (s, 1H), 4.03 (s, 3H), 3.17-3.19 (m, 4H), 2.00-2.17 (m, 4H), 1.77 (s, 3H), (s, 4H), (s, 3H), (s, 1H),

### Example 13—(R)-1-(4-(4-((1-(3-(difluoromethyl)-2-fluorophenyl) ethyl) amino)-7-methoxycinnolin-6-yl) piperazin-1-yl) ethan-1-one

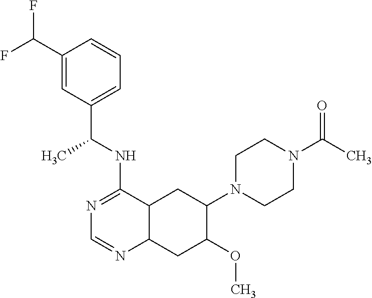 [SMILES: COC1CC2N=CN=C(N[C@H](C)c3cccc(C(F)F)c3)C2CC1N1CCN(C(C)=O)CC1]

1Hnmr: δ 8.15 (s, 1H), 7.68 (s, 1H), 7.53-7.62 (m, 2H), 7.42-7.49 (m, 2H), 7.29-7.30 (m, 2H), 5.23 (t, J=6.4 Hz, 1H), 3.99 (s, 3H), 3.66-3.67 (m, 4H), 3.13-3.19 (m, 4H), 2.08 (s, 3H), 1.68 (d, J=6.4 Hz, 3H).

### Example 14—N—((S)-1-(4-((@-1-(3-(difluoromethyl)-2-fluorophenyl) ethyl) amino)-7-methoxycinnolin-6-yl) 63pyrrolidine-3-yl) acetamide

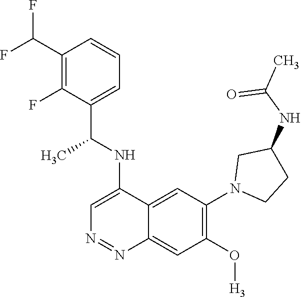

1H nmr: 6 8.20 (d, J=6.8 Hz, 1H), 8.03 (s, 1H), 7.52-7.59 (m, 2H), 7.14-7.31 (m, 3H), 7.34 (s, 1H), 7.12 (s, 1H), 5.18-5.21 (m, 1H), 4.34-4.36 (m, 1H), 3.94 (s, 3H), 3.68-3.74 (m, 2H), 3.50-3.60 (m, 2H), 2.13-2.18 (m, 1H), 1.86-1.91 (m, 1H), 1.84 (s, 3H), 1.67 (d, J=6.8 Hz, 3H)

### Example 15—N—((R)-1-(3-(difluoromethyl)-2-fluorophenyl) ethyl)-7-methoxy-6-(((S)-1-(methyl sulfonyl) pyrrolidin-3-yl)oxy)cinnolin-4-amine

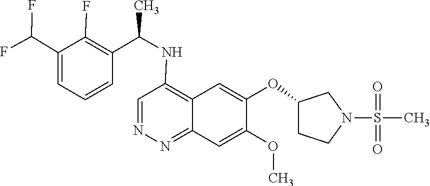 [SMILES: COc1cc2nncc(N[C@H](C)c3cccc(C(F)F)c3F)c2cc1O[C@H]1CCN(S(C)(=O)=O)C1]

1H nmr: 6 8.18 (s, 1H), 7.79 (s, 1H), 7.55-7.62 (m, 2H), 7.54 (s, 1H), 7.39-7.41 (m, 1H), 7.29-7.31 (m, 2H), 5.25 (t, J=6.80 Hz, 1H), 3.94 (s, 3H), 3.73-3.77 (m, 1H), 3.44-3.53 (m, 3H), 2.96 (s, 3H), 2.25-2.29 (m, 2H), 1.70 (d, J=6.8 Hz, 3H).

### Example 16—(R)-1-(4-(4-((1-(3-(difluoromethyl)-2-fluorophenyl)ethyl)amino)-7-methoxycinnolin-6-yl)-3,6-dihydropyridin-1(2H)-yl)ethan-1-one

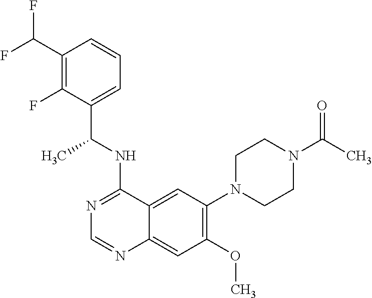 [SMILES: COc1cc2ncnc(N[C@H](C)c3cccc(C(F)F)c3F)c2cc1N1CCN(C(C)=O)CC1]

1H nmr: 6 8.25-8.28 (m, 2H), 7.54-7.62 (m, 2H), 7.41 (s, 1H), 7.25 (t, J=7.6 Hz, 1H), 6.93-7.20 (m, 1H), 6.04-6.05 (m, 1H), 5.33-5.35 (m, 1H), 4.26-4.29 (m, 2H), 4.07 (s, 3H), 3.76-3.83 (m, 2H), 2.59-2.70 (m, 2H), 2.22 (s, 3H), 1.78 (d, J=6.4 Hz, 3H)

### Example 17—N—((R)-1-(3-(difluoromethyl)-2-fluorophenyl) ethyl)-7-methoxy-6-(((S)-1-methylpyrrolidin-3-yl) oxy) cinnolin-4-amine

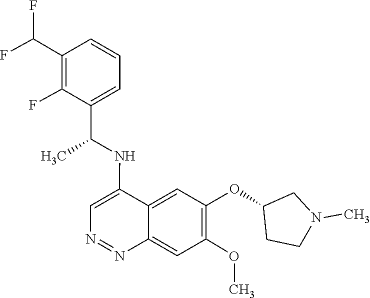 [SMILES: COc1cc2nncc(N[C@H](C)c3cccc(C(F)F)c3F)c2cc1O[C@H]1CCN(C)C1]

1H nmr: 6 8.17 (s, 1H), 7.64 (s, 1H), 7.53-7.59 (m, 2H), 7.44 (s, 1H), 7.38-7.42 (m, 1H), 7.15-7.32 (m, 2H), 5.18-5.26 (m, 2H), 3.94 (s, 3H), 2.99-3.03 (m, 2H), 2.61-2.72 (m, 2H), 2.31 (s, 3H), 1.85-1.92 (m, 2H), 1.69 (d, J=6.8 Hz, 3H).

### Example 18—(R)-1-(4-(4-((1-(3-(difluoromethyl)-2-fluorophenyl) ethyl) amino)-7-methoxycinnolin-6-yl)-4-hydroxypiperidin-1-yl) ethan-1-one

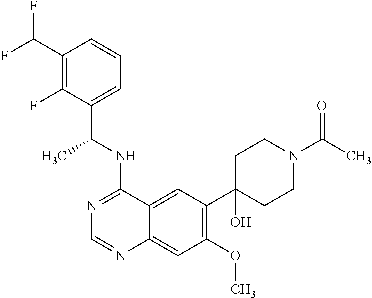 [SMILES: COc1cc2ncnc(N[C@H](C)c3cccc(C(F)F)c3F)c2cc1C1(O)CCN(C(C)=O)CC1]

1H nmr: 6 8.63 (s, 1H), 8.27 (s, 1H), 8.05 (bs, 1H), 7.64-7.67 (m, 1H), 7.54-7.58 (m, 1H), 7.46 (s, 1H), 7.15-7.33 (m, 2H), 5.38 (s, 1H), 5.28-5.31 (m, 1H), 4.34-4.37 (m, 1H), 3.97 (s, 3H), 3.73-3.75 (m, 1H), 3.48-3.54 (m, 1H), 3.00 (t, J=13.2 Hz, 1H), 2.34-2.37 (m, 2H), 2.06 (s, 3H), 1.68 (d, J=6.8 Hz, 3H), 1.58-1.61 (m, 2H).

### Example 19—(R)-1-(4-((1-(3-(difluoromethyl)-2-fluorophenyl) ethyl) amino)-7-methoxycinnolin-6-yl) cyclohexane-1, 4-diol

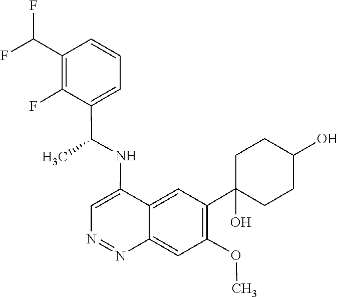 [SMILES: COc1cc2nncc(N[C@H](C)c3cccc(C(F)F)c3F)c2cc1C1(O)CCC(O)CC1]

1H nmr: δ 8.62 (s, 1H), 8.22 (s, 1H), 7.84-7.86 (d, J=6.8 Hz, 1H), 7.63-7.67 (m, 1H), 7.53-7.57 (m, 1H), 7.42-7.49 (m, 1H), 7.15-7.32 (m, 2H), 5.23-5.26 (m, 1H), 4.97 (s, 1H), 4.52 (d, J=4.4 Hz, 1H), 3.97 (s, 3H), 3.52 (bs, 2H), 2.36-2.43 (m, 3H), 1.72-1.78 (m, 2H), 1.67 (d. J=6.4 Hz. 3H), 1.59-1.63 (m, 2H).

### Identification of SOS1 Inhibitors:

Compounds were screened using an HTRF KRAS WT/SOS1 PPI kit provided by Cis bio, according to the manufacturer's instructions, with the modifications described below. All reagents were allowed to equilibrate to room temperature and diluted to working stock in Binding Domain Detection buffer (BDD buffer). Stock solutions were prepared by adding 1 part Tag1-KRAS WT/GTP to 1 part Tag2-SOS1 and 1 part BDD. Separately, 1 part Anti-Tag1 XL665 antibody premix was added to Anti-Tag2 Tb cryptate antibody premix.

BI-3406 (Selleck Chemicals) was used as a standard, solubilised to 10 mM in DMSO. Test compounds were also solubilised to 10 mM in DMSO. Test compounds were then dispensed in a ½-log, 10-point dilution series starting at 10 μM, using a Labcyte Echo 650 instrument to dispense 200 nL of compound per well into 384-well microplates (Greiner 784075). Per well, 6 uL of Tag1-KRAS WT/GTP premix, 4 ul Tag2-SOS1 premix and 10 μl of Anti-Tag1 XL665 and Anti-Tag2 Tb cryptate antibodies were then added using a E1-ClipTip™ Electronic Adjustable Multichannel Equalizer Pipette. After the final addition, the plate was covered and placed in the benchtop incubator set at 22° C. The reaction was incubated for 60 minutes at room temperature, prior to reading using a Pherastar FSX plate reader.

All the compounds of the present invention had an IC₅₀ of less than 500 nM when tested in the assay.

The compound of Example-8: 6-(6-oxa-3-azabicyclo [3.1.1] heptan-3-yl)-N—((R)-1-(3-(difluoromethyl)-2 fluorophenyl) ethyl)-7-methoxycinnolin-4-amine

had an IC₅₀ of <100 nM.

1. A Compound of formula (I) 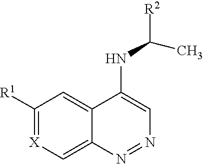
Or a pharmaceutically acceptable salt or solvate thereof, wherein:
X is N or CR⁹
R¹ is selected from:
O—C₁₋₆ alkyl
O—R¹⁰
O—C₁₋₆ alkyl-R10
R¹¹
R² is selected from 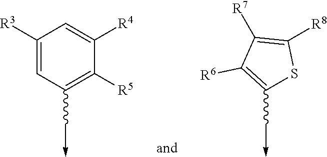
R³ is H or NH₂
R⁴ is CF₃, CF₂CH₂OH, CN
R⁵ is halo
R⁶ is H
R⁷ is H
R⁸ is Ph, ortha substituted by CH₂NHCH₃
R⁹ is H, OCH₃, F and CN
R¹⁰ is 4-6 membered saturated heterocycle containing 1-2 heteroatoms, selected from O, N & S wherein S may also include SO and SO₂ moieties, said heterocycle may also be optionally substituted by C(O)C₁₋₆ alkyl, S(O)₂C₁₋₆ alkyl and C₁₋₆ alkyl,
R¹¹ is 4-6 membered saturated, or partially saturated heterocycle containing 1-2 heteroatoms, selected from O, N & S wherein S may also include SO and SO₂ moieties, said heterocycle may also be optionally substituted by 1-2 groups selected from C(O)C₁₋₆alkyl, N C(O)C₁₋₆ alkyl, OH, an ether linkage to form a bi-cycle and C₁₋₆ alkyl.

2. A compound according to claim 1 wherein R² is 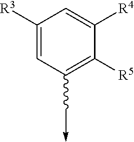
R³ is H or NH₂
R⁴ is CF₃, CF₂CH₂OH, CN
R⁵ is F.

3. A compound according to claim 1 wherein R² is 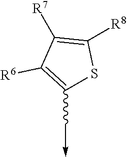
R⁶ is H
R⁷ is H
R⁸ is Ph, ortha substituted by CH₂NHCH₃
R⁹ is H, OCH₃, F and CN.

4. A compound according to any of claims 1-3 wherein R¹ is selected from:
O—C₁₋₆ alkyl, O—R¹⁰, O—C₁₋₆ alkyl-R¹⁰ and R¹¹
Wherein R¹⁰ is selected from: 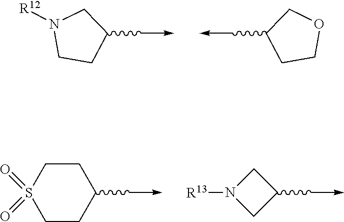
Wherein R¹¹ is selected from: 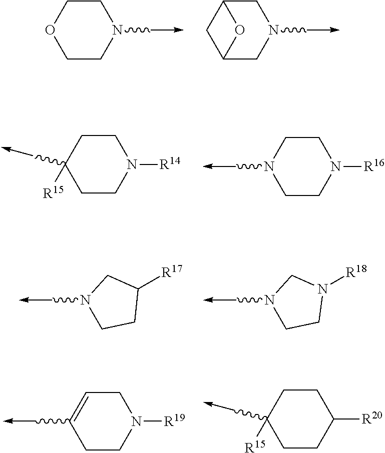
is selected from C(O)C₁₋₆ alkyl, C₁₋₆alkyl, H
R¹³ is selected from C(O)C₁₋₆ alkyl, C₁₋₆alkyl, H
R¹⁴ is selected from C(O)C₁₋₆ alkyl, NC(O)C₁₋₆ alkyl, OH and C₁₋₆ alkyl,
R¹⁵ is OH
R¹⁶ is selected from C(O)C₁₋₆ alkyl, NC(O)C₁₋₆ alkyl, OH and C₁₋₆ alkyl,
R¹⁷ is selected from C(O)C₁₋₆ alkyl, NC(O)C₁₋₆ alkyl, OH and C₁₋₆ alkyl,
R¹⁶ is selected from C(O)C₁₋₆ alkyl, NC(O)C₁₋₆ alkyl, OH and C₁₋₆ alkyl,
R¹⁹ is selected from C(O)C₁₋₆ alkyl, NC(O)C₁₋₆ alkyl, OH and C₁₋₆ alkyl,
R²⁰ is selected from C(O)C₁₋₆ alkyl, NC(O)C₁₋₆ alkyl, OH and C₁₋₆ alkyl.

5. A compound according to claim 4 wherein R¹ is selected from:
O—C₁ alkyl, O—R¹⁰ and O—C₁ alkyl-R¹¹
Wherein R¹⁰ is selected from: 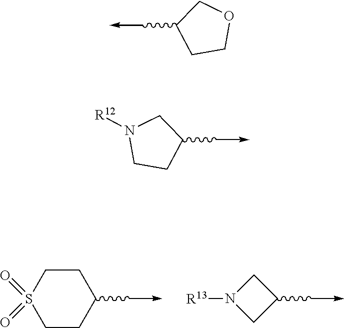
Wherein R¹¹ is selected from 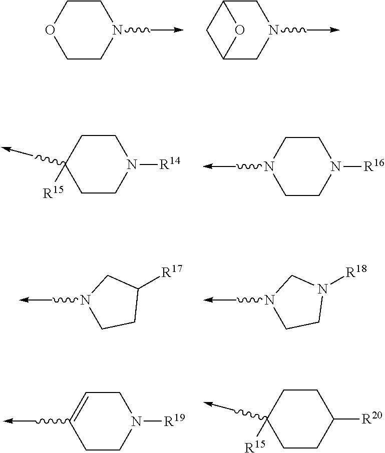
Wherein
R¹² is selected from C(O)CH₃, CH₃ and H,
R¹³ is selected from C(O)CH₃
R¹⁴ is selected from C(O)C₁₋₆ alkyl,
R¹⁵ is OH
R¹⁶ is selected from C(O)C₁₋₆ alkyl,
R¹⁷ is selected from NC(O)C₁₋₆ alkyl,
R¹⁶ is selected from C(O)C₁₋₆ alkyl,
R¹⁹ is selected from C(O)C₁₋₆ alkyl,
R²⁰ is selected from C(O)C₁₋₆ alkyl, NC(O)C₁₋₆ alkyl, OH and C₁₋₆ alkyl.

6. A compound according to any preceeding claim wherein X is N or CR⁹, wherein R⁹ is H.

7. A compound according to any preceeding claim wherein X is CR⁹, wherein R⁹ is H.

8. A compound according to any of claims 1-7 for use as a medicament.

9. A compound according to any of claims 1-7 for use in the treatment of pain.

10. A compound for use according to claim 9 wherein pain includes acute pain, chronic pain, inflammatory pain, nociceptive pain, neuropathic pain, hyperalgesia, allodynia, central pain, cancer pain, post-operative pain, visceral pain, musculo-skeletal pain, heart or vascular pain, head pain, orofacial pain and back pain.

11. A compound according to any of claims 1-7 for use in the treatment of cancer.

12. A compound for use according to claim 11 wherein cancer is selected from the group consisting of pancreatic cancer, lung cancer, colorectal cancer, cholangiocarcinoma, multiple myeloma, melanoma, uterine cancer, endometrial cancer, thyroid cancer, acute myeloid leukaemia, bladder cancer, urothelial cancer, gastric cancer, cervical cancer, head and neck squamous cell carcinoma, diffuse large B cell lymphoma, oesophageal cancer, chronic lymphocytic leukaemia, hepatocellular cancer, breast cancer, ovarian cancer, prostate cancer, glioblastoma, renal cancer and sarcomas.

13. A compound according to any of claims 1-7 for use in the treatment of dementia, including Alzheimers, Mild cognitive impairment (MCI), Creutzfeldt-Jakob disease (CJD), Dementia with Lewy bodies (DLB), Vascular dementia, Alcohol-related brain damage (ARBD), Young-onset dementia, Frontotemporal dementia (FTD) & HIV-related cognitive impairment.

14. A compound according to any of claims 1-7 for use in the treatment of parkinsonism, also known as parkinsons disease, including idiopathic Parkinson's, Vascular parkinsonism (also known as arteriosclerotic parkinsonism), drug-induced parkinsonism, multiple system atrophy, progressive supranucleur palsy, normal pressure hydrocephalus, tremors, including essential tremor & Wilsons disease.
